(19) United States
(12) Patent Application Publication
(10) Pub. No.: US 2006/0115449 A1
(43) Pub. Date: Jun. 1, 2006

(54) Bioabsorbable, biobeneficial, tyrosine-based polymers for use in drug eluting stent coatings

(75) Inventor: Stephen Dirk Pacetti, San Jose, CA (US)

Correspondence Address: SQUIRE, SANDERS & DEMPSEY LLP, 1 MARITIME PLAZA, SUITE 300, SAN FRANCISCO, CA, 94111, US

(73) Assignee: Advanced Cardiovascular Systems, Inc.

(21) Appl. No.: 11/000,572

(22) Filed: Nov. 30, 2004

(51) Int. Cl. A61K 47/48; C08G 63/91; A61K 31/785

(52) U.S. Cl. 424/78.27; 424/423; 525/54.1; 525/54.2

(57) ABSTRACT

This document discloses a family of tyrosine carbonate polymers and polymer mixtures that may contain bioactive or biobeneficial polymers or constituents. Methods of making these polymers and mixtures are disclosed, as well. Also, implantable or partially implantable medical devies constructed with or from these polymers are disclosed.

### BACKGROUND

Percutaneous transluminal coronary angioplasty (PTCA) is a procedure for treating heart disease. A surgeon introduces a catheter assembly having a balloon portion percutaneously into the cardiovascular system of a patient via the brachial or femoral artery. The surgeon advances the catheter assembly through the coronary vasculature until the balloon portion crosses the occlusive lesion. Once in position, the surgeon inflates the balloon to radially compress the atherosclerotic plaque of the lesion and remodel the vessel wall. The surgeon then deflates the balloon to remove the catheter.

But this procedure can tear arterial linings or create intimal flaps, which can collapse and occlude the vessel after balloon removal. Moreover, thrombosis and restenosis of the artery may develop over several months following the procedure, which may require another angioplasty procedure or a by-pass operation. To reduce artery occlusion, thrombosis, and restenosis, the surgeon can implant a stent into the vessel.

Stents are used not only to provide mechanical support, but also to provide biological therapy. Mechanically, stents act as scaffoldings, physically holding open and, if desired, expanding the vessel wall. Typically, stents compress for insertion through small vessels and then expand to a larger diameter once in position. U.S. Pat. No. 4,733,665, issued to Palmaz; U.S. Pat. No. 4,800,882, issued to Gianturco; and U.S. Pat. No. 4,886,062, issued to Wiktor disclose examples of PTCA stents.

Medicating the stent provides for biological therapy. Medicated stents allow local drug administration at the diseased site. To provide an effective drug concentration at the treated site, systemic treatment often requires concentrations that produce adverse or toxic effects. Local delivery advantageously allows for smaller systemic drug levels in comparison to systemic treatment. Because of this, local delivery produces fewer side effects and achieves results that are more favorable. One proposed method for medicating stents involves coating a polymeric carrier onto a stent surface. This method applies a solution that includes a solvent, a dissolved polymer, and a dissolved or dispersed drug to the stent. As the solvent evaporates, it leaves a drug impregnated, polymer coating on the stent.

Current biomaterials research aims at controlling protein adsorption on implantable medical devices. Currently implanted materials can exhibit uncontrolled protein adsorption, leading to a mixed layer of partially denatured proteins. Protein coated surfaces contain different cell binding sites resulting from adsorbed proteins such as fibrinogen and immunoglobulin G. Platelets and inflammatory cells such as macrophages and neutrophils adhere to these surfaces. When so activated, these cells secret a wide variety of pro-inflammatory and proliferative factors. Non-fouling surfaces control these events. These surfaces absorb little or no protein, primarily due to their hydrophilicity. One prior art approach creates these surfaces by using hyaluronic acid or polyethylene glycol. Non-fouling surfaces or coatings are a subset of biobeneficial coatings. Biobeneficial coatings benefit the treatment site without releasing pharmaceutically or therapeutically active agents, (“drug(s)”). Another type of biobeneficial coating contains free-radical scavengers, which preserve nitric oxide and prevent oxidative damage. Yet another type of biobeneficial coating contains agents that catalytically produce nitric oxide from endogenous species.

Biobeneficial coatings are surfaces that are intended to have a biological benefit without the release of pharmaceutically active agents. The world of biobeneficial coatings may be divided into two categories, those that are intended to bioabsorb and those that are intended to be biostable. Desirable properties for bioabsorbable, biobeneficial coatings include any of the following properties:

1. Improved bioactivity in-vitro and in-vivo

measured by platelet adhesion,

protein binding,

inflammatory response, etc.

2. Improved mechanical properties

minimal cracking on stent expansion
substantially withstands stent catheter attachment processes. These processes involve crimping or other steps that apply heat and pressure to the stent-balloon segment.
substantially withstands the shear of the balloon during stent deployment
   3. Improved bioabsorption rate
should degrade slowly enough to minimize inflammatory response
should degrade slowly enough to capture some biobeneficial benefit
should degrade fast enough to complete degradation in an accessible time (preferably less than around 6 months)

Tyrosine-based bioabsorbable polymers have the advantages of tunability of mechanical properties and bioabsorption rate. The aromatic tyrosine dipeptide increases rigidity in the polymer backbone, raising the T_{g} for good mechanical strength. It is also amorphous, which improves solvent solubility, precludes the existence of polymer crystallites, and increases absorption rate predictability.

### SUMMARY

Invention polymers comprise mixtures of A-moieties, B-moieties, C-moieties, and D-moieties, which are defined below. It should be understood that invention polymers have at least one A-moiety. Moreover, for those embodiments that have optional B-moieties, C-moieties, or D-moieties, embodiments exist that have two or more different A-moieties, two or more different B-moieties, two or more different C-moieties, or two or more different D-moieties. Furthermore, some embodiments can be chosen to specifically exclude one of or any combination of B-moieties, C-moieties, or D-moieties.

A more general description of some polymer embodiments arises by defining the polymer to comprise at least one A-block comprising one or more A-moieties with the following formula,

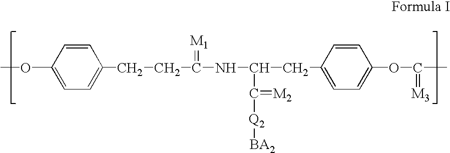

and at least one B-block comprising one or more B-moieties with the following formula,

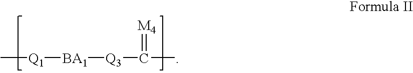

M₁-M₄ can be independently chosen from the following: O, NH, CH₂, or S. In some embodiments, M₁-M₄ can be independently chosen from O or NH.

Q₁-Q₃ can be independently chosen from Group-15- or Group-16-containing moieties, or alternatively, N-, O-, S-, P-, or Se-containing moieties, or alternatively, N- or O-containing moieties, such as NH, NR′, or O wherein R′ is a C₁-C₂₀, linear or branched, (un)substituted alkyl or aryl.

BA₁ and BA₂ can be independently chosen from R-groups (C₁-C₂₀, linear or branched, (un)substituted alkyls or aryls), or a bioactive moiety, provided that 100% of both BA₁ and BA₂ cannot be an R-group. The broadest class of bioactive moieties comprises at least one substituent that provides or causes a biological effect.

Invention polymers can optionally comprise a C-moiety comprising at least one diol. Diols (C-moieties) are organic molecules that contain two alcoholic functionalities, have from 2-30 carbon atoms, and can be (un)branched or (un)substituted. Some embodiments select the diols from those molecules comprising 3-12 carbon atoms. In the diol structure shown in Formula VII, below, R₃ has from 1-20 carbon atoms, if it is present in the polymer.
—O—R₃—O— Formula III

In some embodiments, amine terminated C-moieties are also possible. However, C-moieties can also be any linear or branched diamine with 2 to 16 carbon atoms.

Invention polymers can optionally comprise at least one D-moiety that is a diacid, as shown below in Formula VIII. Diacids (D-moieties) are organic molecules that contain two carboxylic acid functionalities and have from 2-30 carbon atoms. The diacids can be (un)branched or (un)substituted. In some embodiments, diacids can include any one of or any combination of 2-30 carbon atom, (un)branched, (un)substituted diacids. Also, for purposes of this disclosure, diacids also encompass diacid chlorides and molecules that terminate with an acid functionality at one end and an acid chloride functionality at the other end. In the diacid structure below, R₂ has from 1-20 carbon atoms, if it is present in the polymer.

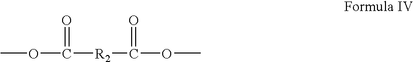

The invention polymers are used to prepare medical devices either predominately constructed with the polymers or medical devices in which the polymer is a more minor constituent, such as a coating or film. In some embodiments, the medical devices are implantable or compose implantable structures. In some embodiments, the medical device is a stent.

Also, methods of making invention polymers are disclosed.

### DETAILED DESCRIPTION

A “non-fouling moiety” is a portion of a chemical compound that is capable of providing the compound the ability to prevent, or at least reduce, the build-up of a denatured layer of protein on the stent surface or on the stent coating. It is a type of bioactive and a type biobeneficial moiety.

“Biobeneficial coatings” benefit the treatment site without releasing pharmaceutically or therapeutically active agents, (drug(s)).

“Biodegradable” means that a substance is hydrolytically labile, oxidatively labile, or susceptible to enzymatic action and is intended to be substantially broken down by the in vivo environment in an amount of time of from 1 to 24 months; alternatively, in an amount of time of from 2 to 18 months; alternatively, in an amount of time of from 3 to 12 months. For purposes of this disclosure, substantially broken down means that non-invasive diagnostic procedures as skilled artisans normally employ cannot detect the polymer in vivo. The in vivo degradation process can be mimicked in vitro in several ways. By aging the device with degradable material at 37° C. in phosphate buffered saline at pH 7.4, the hydrolytic processes may be reproduced. If oxidative mechanisms are relevant then the same solution may be supplemented with oxidants such as hydrogen peroxide or superoxide salts. Additionally, if enzymatic degradation processes are important, representative enzymes can be added to the solution. It is to be understood that while such in vitro tests can mimic the chemical processes operant in vivo, they predict kinetics and rates inaccurately. The term “non-fouling complex” refers to polymeric substances that comprise a non-fouling moiety.

“Unbranched” means that a polymer has less than 0.1 mole percent of sidechains having more than 10 atoms; alternatively, less than 0.01 mole percent of such sidechains; alternatively, less than 0.001 mole percent of such sidechains.

“Branched” means that a polymer has greater than 0.1 mole percent of sidechains having more than 10 atoms; alternatively, greater than 0.01 mole percent of such sidechains; alternatively, greater than 0.001 mole percent of such sidechains.

“Uncrosslinked” means that a polymer sample contains less than 0.1 mole percent of cross-linked polymers; alternatively, invention polymers have less than 0.01 mole percent of cross-linked polymers; alternatively, invention polymers have less than 0.001 mole percent of cross-linked polymers.

“Crosslinked” means that a polymer sample contains greater than 0.1 mole percent of connections between two polymer chains; alternatively, greater than 0.01 mole percent connections between two polymer chains; alternatively, greater than 0.001 mole percent of connections between two polymer chains.

“Partially cross-linked” means having greater than 0.001 mole percent and less than 0.1 mole percent of cross-linked polymers.

“Hydrolytically unstable” or “unstable to hydrolysis” are defined as the characteristic of a compound (e.g., a polymer or a polymeric adduct) when exposed to aqueous fluids having near neutral pH (e.g., blood), to be substantially hydrolyzed within 0 to 24 months, 0 to 12 months, 0 to 6 months, or 0 to 1 month. The temperature of an aqueous liquid to which a compound is exposed can be between room temperature and about 37 C.

“Substantially hydrolyzed” is defined as losing 95 or more percent, 75 or more percent, 50 or more percent, 40 or more percent, or 20 or more percent of the polymer (by mass) to hydrolysis.

One way of determining whether a polymer or a polymeric adduct is hydrolytically stable includes (a) depositing the polymer or adduct on a stent to make a polymer-coated stent; (b) weighing the polymer-coated stent; (c) immersing the polymer-coated stent into an aqueous fluid having near neutral pH; and (d) periodically weighing the stent. If after exposure for enough time to meet the above time definition, little enough polymer or adduct remains on the stent to meet the above mass definitions, the polymer or adduct is defined as “hydrolytically unstable.”

Depending upon the reaction sequence and relative reactivity of the component monomers, invention polymers can be chosen to be more random-like or more block-like. Sometimes, the degree of “randomness” or “blackness” is generically referred to as polymer topology. For purposes of this disclosure, a polymer is characterized as having a more random-like topology if the number of matching adjacent A-moieties, B-moieties, C-moieties, or D-moieties is small, such as less than 50% for at least one or at least two of these moieties or such as less than 35% for at least one or at least two of these moieties; for purposes of this disclosure, a polymer has a random topology if at least one or at least two of these moieties has less than 25% or 10% matching adjacent moieties. For purposes of this disclosure, a polymer is characterized as having a more block-like topology if the number of matching adjacent A-moieties, B-moieties, C-moieties, or D-moieties is large, such as greater than 50% for at least one or at least two of these moieties or such as greater than 60% for at least one or at least two of these moieties for purposes of this disclosure, a polymer has a block topology if at least one or at least two of these moieties has greater than 75% or 90% matching adjacent moieties. For purposes of this disclosure the phrase “greater than X % matching adjacent moieties” means that a given moiety, i.e. an A-moiety, B-moiety, C-moiety, or D-moiety, has an X % chance of being next to another of its kind. For example, A-moiety with 50% matching adjacent moieties would on average be connected to one other A-moiety. For purposes of this disclosure, two or more joined A-moieties, two or more joined B-moieties, two or more joined C-moieties, or two or more joined D-moieties are sometimes referred to as A-blocks, B-blocks, C-blocks, or D-blocks, respectively.

If the particular discussion of a polymer is silent regarding polymer topology, that discussion encompasses embodiments with polymer topology selected from all topologies, random-like topologies, block-like topologies, random topologies, block topologies, and topologies intermediate between random-like and block-like topologies. Moreover, in some embodiments the polymer is selected to exclude polymers with topologies selected from random-like, block-like, random, block, topologies intermediate between random-like and block-like, or any combination of these topologies.

Throughout this disclosure, phenyl or benzyl rings are referred to or depicted. Such reference or depiction includes variations in which the phenyl or benzyl rings are additionally substituted at least at the 2, 3, 5, or 6 positions or any combination of these positions. Any substitution is allowed.

What is disclosed is a family of bioabsorbable, non-fouling (biobeneficial) tyrosine-based polymers for use in drug eluting stent DES coatings. More broadly, the polymer in accordance with this invention composes medical devices. This family is composed of several polymer embodiments that will be described separately. Invention polymers can be used structurally for medical devices. In some embodiments, these polymers are used in coatings for medical devices.

Poly(Ether Carbonate)(Tyrosine or Tyrosine Adduct)-Bioactive Moiety Copolymer/Poly(Imine Carbonate) (Tyrosine or Tyrosine Adduct)-Bioactive Moiety Copolymers

Invention polymers can generally be described as containing at least one A-moiety and at least one other B-moiety, C-moiety, or D-moiety. Additionally, these moieties can optionally be linked by a T-moiety. Each of these is described below.

T-moieties are the same or different, optional, biocompatible polymeric or non-polymeric linkage comprising from 1-10,000 atoms. A-moieties, B-moieties, C-moieties, and D-moieties are defined below. It should be understood that invention polymers have at least one A-moiety. Moreover, embodiments exist that have two or more different A-moieties, two or more different B-moieties, two or more different C-moieties, or two or more different D-moieties. Furthermore, some embodiments can be chosen to specifically exclude any one or any combination of B-moieties, C-moieties, or D-moieties.

A more general description of some polymer embodiments arises by defining the polymer to comprise at least one A-block comprising one or more A-moieties with the following formula,

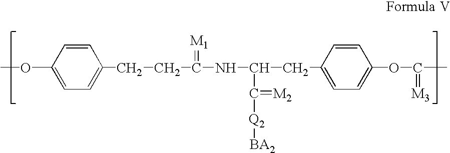

and at least one B-block comprising one or more B-moieties with the following formula,

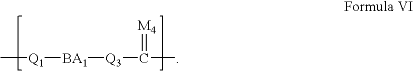

A-moieties or A-blocks are sometimes represented by [A] in formulas throughout this document. For instances in which the polymer has more than one, for example, A-moiety or A-block, the second and subsequent A-moiety or -moieties are sometimes represented by appending one or more “prime” symbols. Thus, [A′] represents a second A-block or -moiety, different from the first.

M₁-M₄ can be independently chosen from the following: O, NH, CH₂, or S. In some embodiments, M₁-M₄ can be independently chosen from O or NH.

Q₁-Q₃ can be independently chosen from Group-15- or Group-16-containing moieties, or alternatively, N-, O-, S-, P-, or Se-containing moieties, or alternatively, N- or O-containing moieties, such as NH, NR′, or O wherein R′ is a C₁-C₂₀, linear or branched, (un)substituted alkyl or aryl.

BA₁ and BA₂ can be independently chosen from R-groups (C₁-C₂₀, linear or branched, (un)substituted alkyls or aryls), or a bioactive moiety, provided that 100% of both BA₁ and BA₂ cannot be an R-group. The broadest class of bioactive moieties comprises at least one substituent that provides or causes a biological effect. Exemplary bioactive moieties can be independently chosen from the following: polyethylene glycol (PEG), poly(propylene glycol) (PPG), poly(tetramethylene glycol), dihydroxy polyvinylpyrrolidone (PVP), dihydroxy poly(styrene sulfonate) (HPSS), poly(2-hydroxyethyl methacrylates) (PHEMA), poly(3-hydroxypropyl methacrylates), poly(3-hydroxypropyl methacrylamide) (PHPMA), poly(alkoxy methacrylates), poly(alkoxy acrylates), polyarginine peptides (PAP), such as R7, phosphoryl choline (PC), dextran, dextrin, sulfonated dextran, dermatan sulfate, heparin (HEP), chondroitan sulfate, glycosaminoglycans, chitosan, sodium hyaluronate, or hyaluronic acid (HA).

Some embodiments constrain BA₂ to greater than 1 mole % bioactive moiety, alternatively, to less than 99 mole % bioactive moiety. Alternatively, some embodiments constrain BA₂ to greater than 10 mole % bioactive moiety and less than 90 mole % bioactive moiety, or greater than 30 mole % bioactive moieties and less than 80 mole % bioactive moieties.

Some embodiments constrain BA₁ to greater than 1 mole % bioactive moiety, alternatively, to less than 99 mole % bioactive moiety. Alternatively, some embodiments constrain BA₁ to greater than 10 mole % bioactive moiety and less than 90 mole % bioactive moiety; or greater than 30 mole % bioactive moieties and less than 80 mole % bioactive moieties.

The selection of BA₁ and BA₂, in some embodiments, can be carried out to exclude any one of or any combination of PEG, PVP, HPSS, PAP, PC, HEP, PPG, poly(tetramethylene glycol), PHEMA, poly(3-hydroxypropyl methacrylates), PHPMA, poly(alkoxy methacrylates), poly(alkoxy acrylates), polyarginine peptides (PAP), such as R7, phosphoryl choline (PC), dextran, dextrin, sulfonated dextran, dermatan sulfate, heparin (HEP), chondroitan sulfate, glycosaminoglycans, chitosan, sodium hyaluronate, or hyaluronic acid (HA). In some embodiments, the selection of M₁-M₄ can be carried out to exclude any one of or any combination of C, NH, CH₂, or S. In some embodiments, the selection of Q₁-Q₃ can be carried out to exclude Group-15- or Group-16-containing moieties; in some embodiments, the selection of Q₁-Q₃ can be carried out to exclude any of or any combination of N-, O-, S-, P-, or Se-containing moieties. Alternatively, in some embodiments the selection of Q₁-Q₃ can be carried out to exclude any of or any combination of N- or O-containing moieties, such as NH, NR′, or O.

Invention polymers can optionally comprise a C-moiety comprising at least one C-moiety that is a diol. Diols (C-moieties) are organic molecules that contain two alcoholic functionalities, have from 2-30 carbon atoms, and can be (un)branched or (un)substituted. Some embodiments select the diols from those molecules comprising 3-12 carbon atoms. In some embodiments, the selection of diols is carried out to exclude any one of or any combination of (un)branched, (un)substituted, C₂-C₃₀ diols. In some embodiments, diols can be independently chosen from ethylene glycol, 1,2-propanediol, 1,3-propanediol, 1,4-butanediol, 1,5-pentanediol, 1,6-hexanediol, 1,7-heptanediol, 1,8-octanediol, 1,9-nonanediol, 1,10-decanediol, 1,11-undecanediol, and 1,12-dodecanediol. In some embodiments, the diol is 1,4-butanediol. In the diol structure shown in Formula VII, below, R₃ has from 1-20 carbon atoms, if it is present in the polymer.
—O—R₃—O— Formula VII

Amine terminated C-moieties are also possible. Preferred amino terminated moieties are 1,2-ethanediamine, 1,4-butanediamine (putrescine) and 1,5-pentanediamine (cadaverene). However, C-moieties can also be any linear or branched diamine with 2 to 16 carbon atoms.

Invention polymers can optionally comprise at least one D-block comprising at least one D-moiety that is a diacid, as shown below in Formula VIII. Diacids (D-moieties) are organic molecules that contain two carboxylic acid functionalities and have from 2-30 carbon atoms. The diacids can be (un)branched or (un)substituted. In some embodiments, diacids can include any one of or any combination of 2-30 carbon atom, (un)branched, (un)substituted diacids. Also, for purposes of this disclosure, diacids also encompass diacid chlorides and molecules that terminate with an acid functionality at one end and an acid chloride functionality at the other end. In some embodiments, diacids can be independently chosen from oxalic acid, malonic acid, succinic acid, glutaric acid, adipic acid, pimelic acid, suberic acid, azelaic acid, and sebacic acid. In some embodiments, the selection of diacids can be carried out to exclude any one of or any combination of oxalic acid, malonic acid, succinic acid, glutaric acid, adipic acid, pimelic acid, suberic acid, azelaic acid, or sebacic acid. In some embodiments, the diacid can be selected from sebacic acid, adipic acid, and succinic acid. In the diacid structure below, R₂ has from 1-20 carbon atoms, if it is present in the polymer.

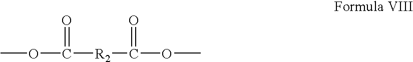

Formula IX, below, depicts a general form of invention polymers showing an A-moiety and a B-moiety combined. This embodiment comprises no T-moieties intervening between the A- and B-moieties.

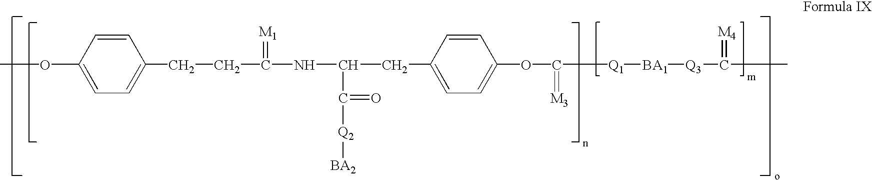

| | | | |
|---|---|---|---|
| o is | 20 to 6000 | or | 40 to 2000 |
| m is | 0.01 to 0.99 | or | 0.05 to 0.95 |
| n is | 0.01 to 0.99 | or | 0.05 to 0.95 |

The variables n and m represent the mole fraction of the A-moiety and the B-moiety; variable o represents the average molecular mass of the polymer. Formula VII shows all of the A-moieties and all of B-moieties as being connected to each other respectively. But throughout this disclosure this representation and similar representations disclose any combination of A-moiety and B-moiety (or with the necessary changes C-moieties or D-moieties) i.e. completely random through completely block-like.

Poly(Ether Carbonate) Tyrosine-Bioactive-Moiety Copolymer

A subset of invention polymers in which M₁-M₄ and Q₁-Q₃, from Formula IX, above, are oxygen can be described as having at least one A-moiety,

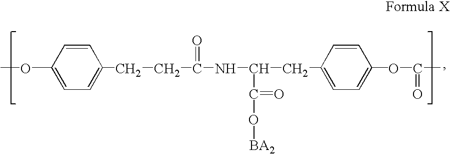

and at least one B-moiety,

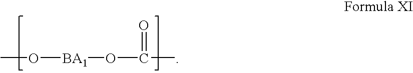

Formula XII, below, depicts an embodiment with an A-moiety and a B-moiety combined and with BA₂ selected to be an R-group.

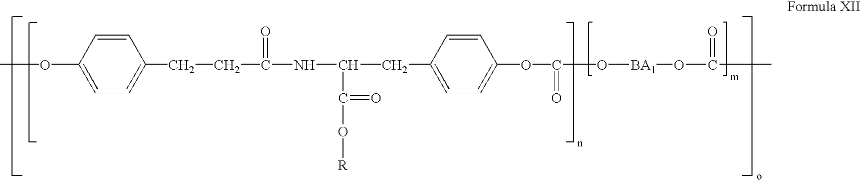

| | | | |
|---|---|---|---|
| o is | 20 to 6000 | or | 40 to 2000 |
| m is | 0.01 to 0.99 | or | 0.05 to 0.95 |
| n is | 0.01 to 0.99 | or | 0.05 to 0.95 |

The mechanical properties can be adjusted by (1) varying the molecular weight of the BA₁ or BA₂, (2) by varying the ratio of tyrosine dipeptide to BA₁ or BA₂, and (3) by varying the R group when BA₁ or BA₂ is an R-group. For embodiments where some of BA₁ or BA₂ is selected to be an R-group, ethyl would be an especially suitable R-group because it cleaves to give ethanol, and such derivatives have been shown to be very biocompatible. K. James, et al. Biomaterials, 20, 2203-2212, 1999. K. Hooper, et al. J. Biomed. Mater. Res, 41, 443-454, 1998. In-vivo, these polymers are expected to be amorphous, but with good mechanical properties. The carbonate linkages can be formed using phosgene, which is very hazardous. They can also be formed with triphosgene or diphosgene, which are considerably less toxic, but more expensive. Consequently, phosgene is cost effective for large scale, industrial synthesis, while triphosgene and diphosgene are useful for small lab scale and custom synthesis. Yet, another synthetic route to the polycarbonate is to use diphenyl carbonate instead of phosgene. This process is done in the melt under vacuum with lithium hydroxide catalyst, and is thermodynamically driven by distilling away phenol. It represents a safe way of producing polycarbonates in the lab, but requires higher temperatures and longer reaction times. Useful temperatures can range from 60° C. to 182° C. Useful reaction times can range from 0.5 to 24 hours.

In the following embodiment, 80-100 percent, or 95-100 percent, of BA₁ can be selected to be an R-group. Any remaining BA₁ and BA₂ can be independently chosen to be PEG.

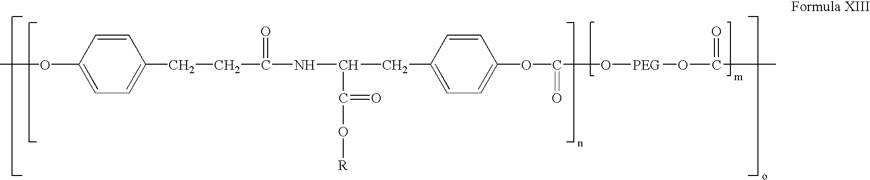

The synthesis of the above poly(ether carbonate) is straightforward. The desaminotyrosyl-tyrosine dipeptide can be combined with the PEG in methylene chloride and phosgene can be added as a solution in toluene. The reaction would be completed in around 9 minutes. In some embodiments, this reaction is carried out for from 1-60 minutes.

Poly(Tyrosine Carbonate) Pendant Bioactive Moiety Groups

In other polymer embodiments, the polymers can be defined by choosing 1% to 75%, alternatively, 5% to 50%, of BA₂ from Formula IX, above, to be a bioactive moiety, as described above. Some of these polymer embodiments can be defined by choosing 1% to 75%, more narrowly 5% to 50%, of Q₂ to be NH and the remainder of Q₂ to be O.

This subset of invention polymers can be described as having at least two A-moieties.

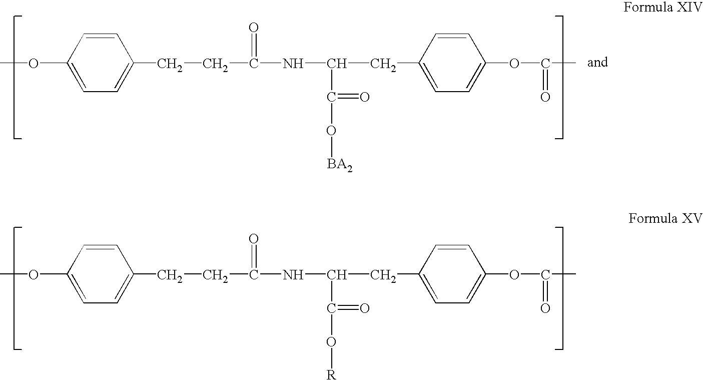

Formula XVI, below, depicts a general form of invention polymers showing two different A-moieties.

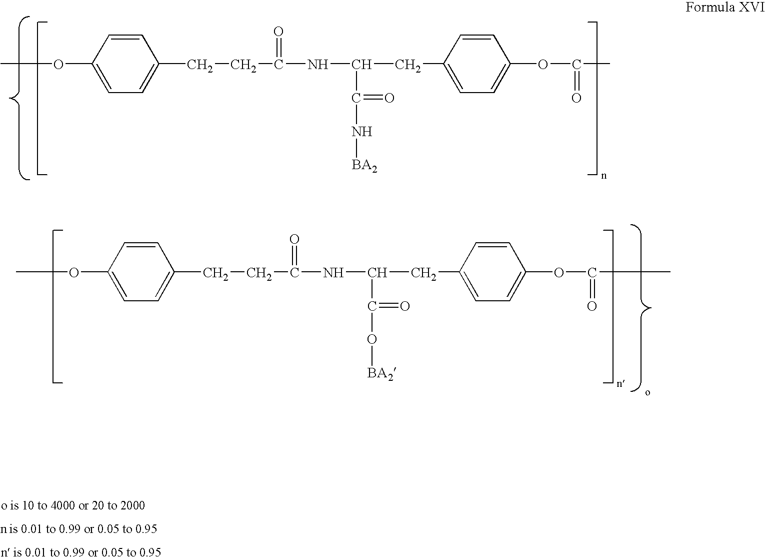

An invention embodiment defined by BA₂ being partially selected to be PEG or a PEG derivative pendant from A-moieties is shown below; the structure shows a tyrosine-derived polycarbonate where some of BA₂ are m-PEG, with some of the rest being R-groups, as described above.

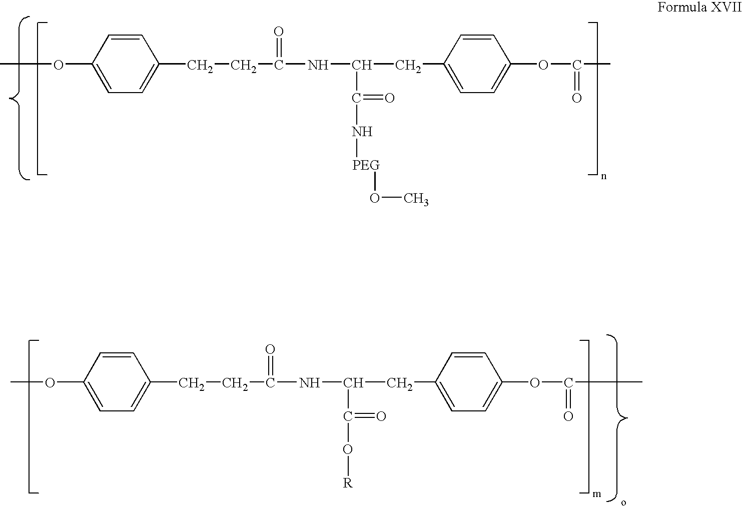

| | | | |
|---|---|---|---|
| o is | 4 to 3000 | or | 10 to 2000 |
| m is | 0.25 to 0.99 | or | 0.50 to 0.95 |
| n is | 0.01 to 0.75 | or | 0.05 to 0.50 |

In embodiments defined by all or substantially all of BA₂ being PEG or a PEG derivative, depending on the molecular weight of the PEG, the polymer could be water soluble. The m-PEG can be added either after the polymerization or as part of a monomer.

Synthesizing the desaminotyrosyl-tyrosine dipeptide with a pendant m-PEG group is straightforward, and can be illustrated by Reaction Scheme I, below.

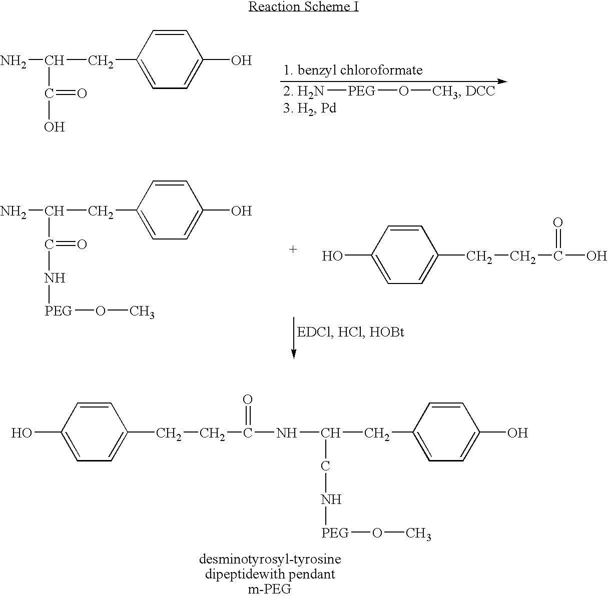

In the embodiment shown in Formula XVIII, below, 0.1% to 50% of BA₂ can be hyaluronic acid (HA). In other embodiments, 2% to 40%, or 5% to 25%, of BA₂ can be HA.

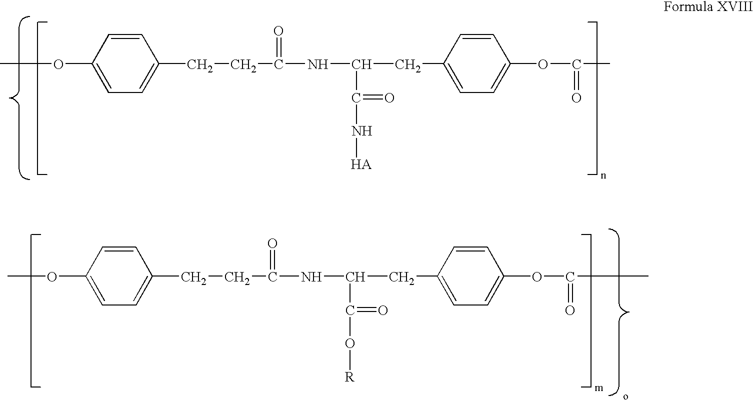

| | | | |
|---|---|---|---|
| o is | 4 to 3000 | or | 10 to 2000 |
| m is | 0.5 to 0.995 | or | 0.75 to 0.99 |
| n is | 0.005 to 0.5 | or | 0.01 to 0.25 |

In the embodiment shown in Formula XIX, below, 1% to 75% of BA₂ can be polyvinylpyrrolidone (PVP). In other embodiments 2% to 50%, or 5% to 25%, of BA₂ can be PVP.

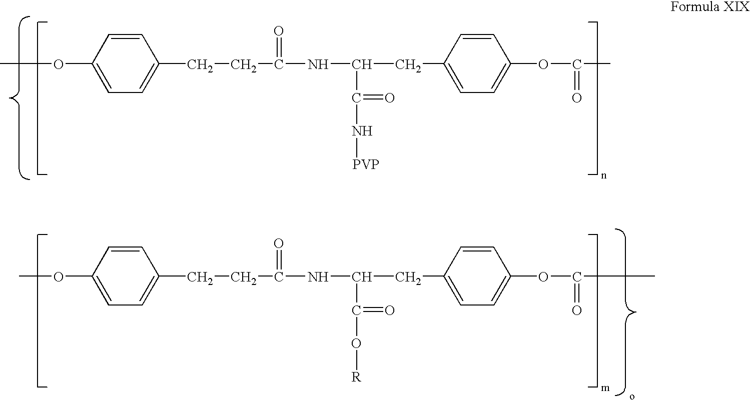

| | | | |
|---|---|---|---|
| o is | 4 to 3000 | or | 10 to 1500 |
| m is | 0.25 to 0.99 | or | 0.50 to 0.95 |
| n is | 0.01 to 0.75 | or | 0.05 to 0.50 |

A particular polymer embodiment, defined as having three separate A-moieties, is shown in Formula XX, below: one A-moiety has BA₂ chosen as PEG or a PEG derivative (such as m-PEG), one A-moiety has BA₂ chosen as HA, and one A-moiety has BA₂ chosen as ethyl.

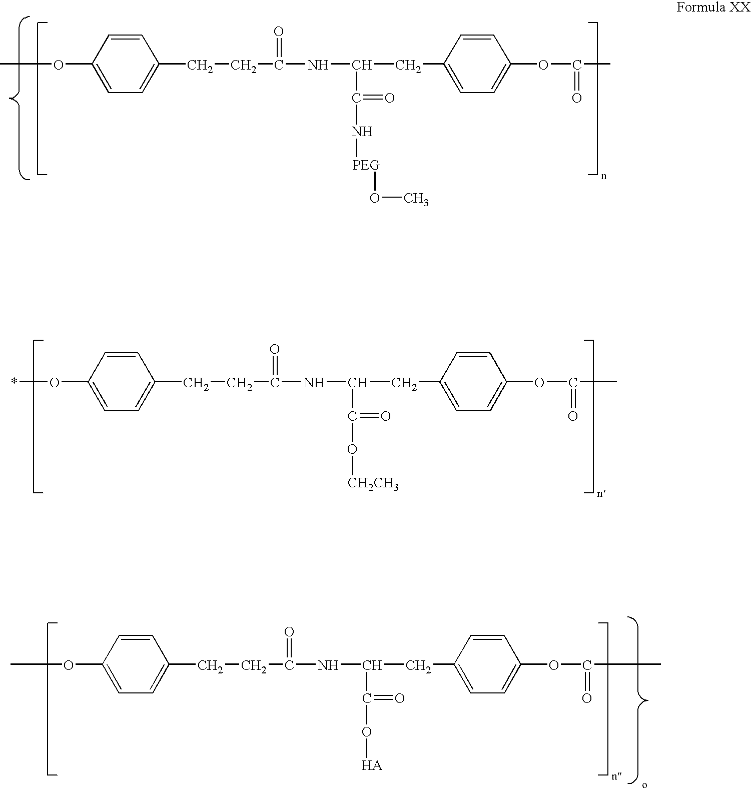

| | | | |
|---|---|---|---|
| o is | 4 to 3000 | or | 10 to 1500 |
| n is | 0 to 0.75 | or | 0.01 to 0.50 |
| n′ is | 0.01 to 0.99 | or | 0.05 to 0.95 |
| n″ is | 0 to 0.55 | or | 0.01 to 0.45 |

Each of these A-moieties can be arranged in any repeat pattern, as is known to those of ordinary skill in the art. The same is true for each of the formulas in this document.

Poly(Ether Carbonate) Tyrosine-Diol Copolymer with Bioactive Moiety in the Backbone

This subset of invention polymers is defined as including an A-moiety, B-moiety, and a C-moiety.

As before, this polymer can have optional T-moieties intervening between the A-, B-, or C-moieties or blocks. T represents the same or different, optional, biocompatible polymeric or non-polymeric linkage comprising from 1-10,000 atoms

This subset of invention polymers can be described as having at least one A-moiety in which all of BA₂ is R. Alternatively, some embodiments of this subset of invention polymers have A-moieties or blocks greater than 90%, or greater than 95%, of BA₂ is R.

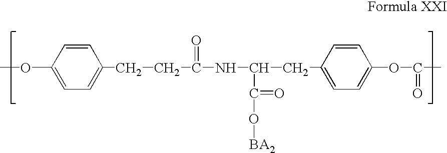

and at least one B-moiety

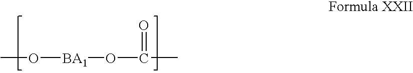

and at least one C-moiety
—O—R₃—O— Formula XXIII

Formula XXIV, below, depicts a general form of invention polymers showing an A-moiety, a B-moiety, and a C-moiety combined. Useful mole percent ranges for BA2 as bioactive moiety are 0 to 90%, or 1% to 75%, or alternatively 5% to 50%.

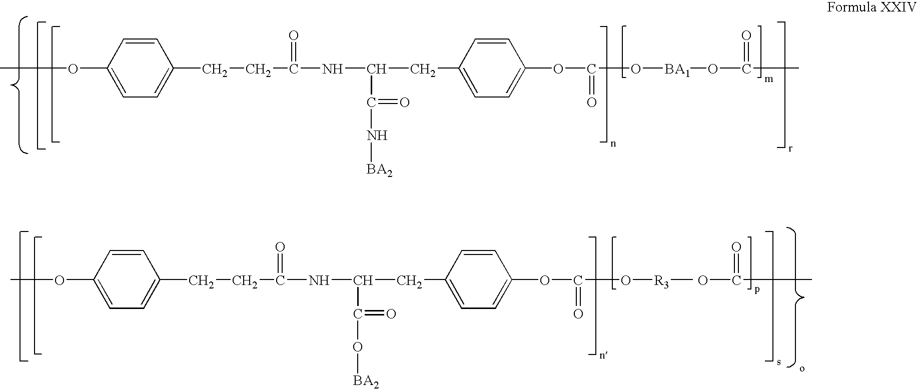

| | | | |
|---|---|---|---|
| m is | 0.1 to 0.99 | or | 0.05 to 0.95 |
| n is | 0 to 0.99 | or | 0.01 to 0.95 |
| o is | 4 to 3000 | or | 10 to 1500 |
| p is | 0.1 to 0.99 | or | 0.05 to 0.95 |
| n′ is | 0 to 0.99 | or | 0.01 to 0.95 |
| r is | 0.01 to 0.75 | or | 0.05 to 0.50 |
| s is | 0.25 to 0.99 | or | 0.50 to 0.95 |

This polymer can be thought of as the tyrosine-carbonate version of POLYACTIVE. POLYACTIVE is a trade name of a polybutylene terephthalate-PEG group of products and is available from IsoTis Corp. of Holland. In various brands of POLYACTIVE, the ratio between the units derived from ethylene glycol and the units derived from butylene terephthalate falls between about 0.67:1 and about 9:1. The molecular weight of the units derived from ethylene glycol can be between about 300 and about 4,000 Daltons. Some embodiments choose 1,4-butanediol because it is used in POLYACTIVE. This polymer could be synthesized in a two-step process to make it more moiety-like, or with all diols reacted at once, which is more random.

An example structure with 1,4-butanediol is shown below in Formula XXV. Useful mole percent ranges for BA2 as bioactive moiety are 0% to 90%, or 1% to 75%, or alternatively 5% to 50%.

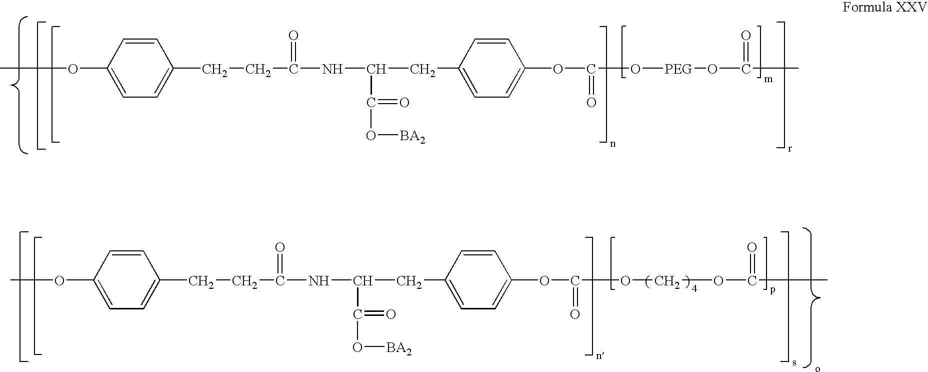

| | | | |
|---|---|---|---|
| m is | 0.01 to 0.80 | or | 0.05 to 0.50 |
| n is | 0 to 0.99 | or | 0.01 to 0.95 |
| o is | 4 to 3000 | or | 10 to 1500 |
| p is | 0.01 to 0.99 | or | 0.05 to 0.95 |
| n′ is | 0.01 to 0.90 | or | 0.05 to 0.75 |
| r is | 0.01 to 0.80 | or | 0.05 to 0.50 |
| s is | 0.005 to 0.995 | or | 0.01 to 0.95 |

Poly(Ether Carbonate) Tyrosine-Diol Copolymer with Pendant Bioactive Moiety

Another subset of invention polymers can be described as having at least one A-moiety.

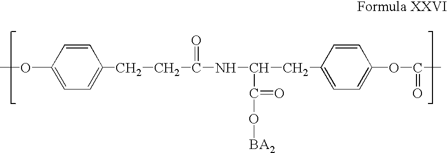

and at least one B-moiety

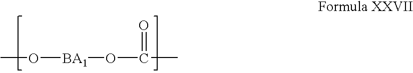

and at least one C-moiety
—O—R₃—O— Formula XXVIII

Useful mole percent ranges for BA2 as bioactive moiety are 0% to 90%, or 1% to 75%, or alternatively 5% to 50%.

A general structure for this polymer is shown below in Formula XXIX.

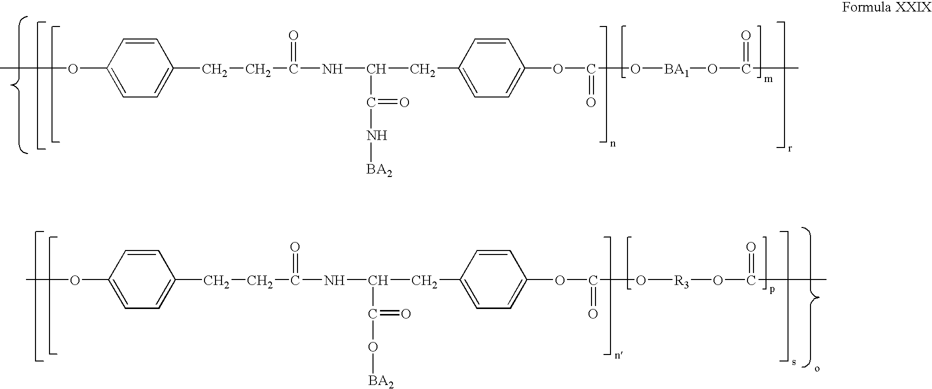

Another embodiment of the polymer depicted above in Formula XXIX is shown below in Formula XXX; BA₁ can be selected to be PEG, BA₂ can independently represent a bioactive moiety, and R₃ can be selected to contain four carbon atoms.

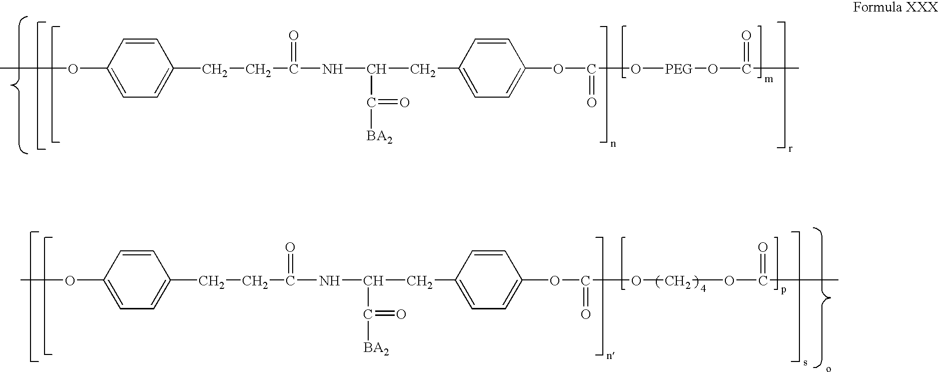

| | | | |
|---|---|---|---|
| m is | 0.01 to 0.80 | or | 0.05 to 0.50 |
| n is | 0 to 0.99 | or | 0.01 to 0.95 |
| o is | 4 to 3000 | or | 10 to 1500 |
| p is | 0.01 to 0.99 | or | 0.05 to 0.95 |
| n′ is | 0.01 to 0.90 | or | 0.05 to 0.75 |
| r is | 0.01 to 0.8 | or | 0.05 to 0.50 |
| s is | 0.005 to 0.995 | or | 0.01 to 0.95 |

An embodiment with two A-moieties, two B-moieties, and two C-moieties is shown below as Formula XXXI.

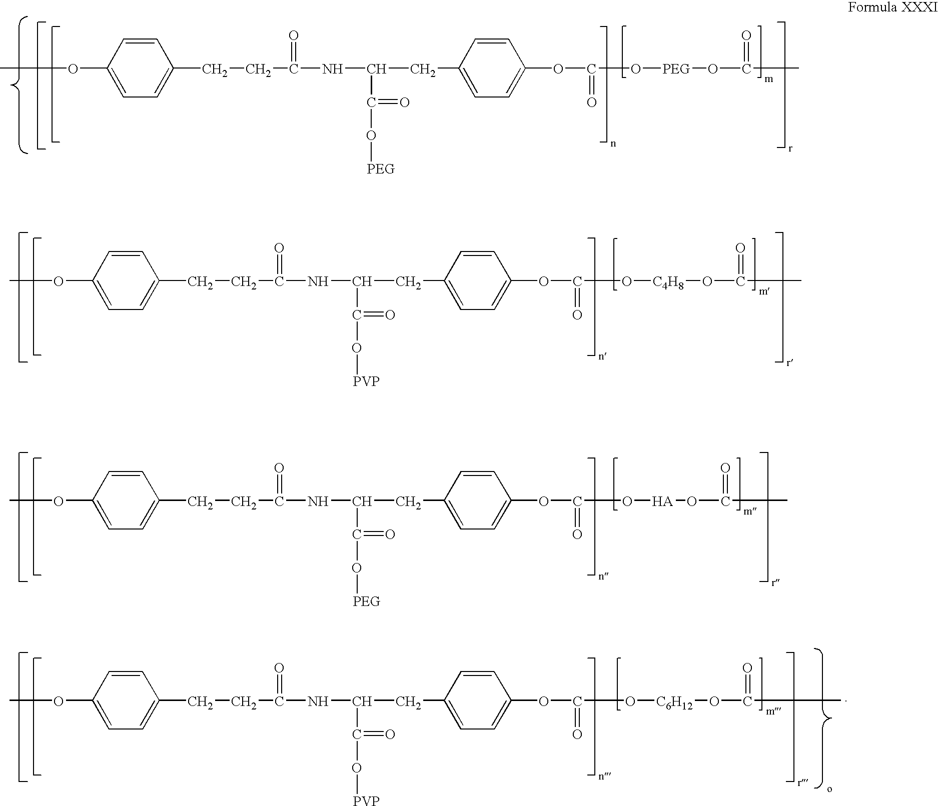

BA₂ in the first A-moiety is PEG; BA₂ in the second A-moiety is PVP. Next, BA₁ in the first B-moiety is PEG; BA₁ in the second B-moiety is HA. Finally, the R group for the first C-moiety is C₄H₈; the R group for the second C-moiety C₆H₁₂.

| | | | |
|---|---|---|---|
| m is | 0.01 to 0.75 | or | 0.05 to 0.50 |
| m′ is | 0.005 to 0.995 | or | 0.01 to 0.99 |
| m″ is | 0.001 to 0.50 | or | 0.005 to 0.25 |
| m′′′ is | 0.005 to 0.995 | or | 0.01 to 0.99 |
| n is | 0.01 to 0.75 | or | 0.05 to 0.50 |
| n′ is | 0.005 to 0.55 | or | 0.01 to 0.45 |
| n″ is | 0.01 to 0.75 | or | 0.05 to 0.50 |
| n′′′ is | 0.005 to 0.55 | or | 0.01 to 0.45 |
| r is | 0.01 to 0.75 | or | 0.05 to 0.50 |
| r′ is | 0.01 to 0.55 | or | 0.02 to 0.45 |
| r″ is | 0.01 to 0.75 | or | 0.05 to 0.50 |
| r′′′ is | 0.01 to 0.55 | or | 0.02 to 0.45 |
| o is | 2 to 3000 | or | 5 to 1500 |

Poly(Ether Ester) Tyrosine-Bioactive-Moiety-Diacid Copolymer

In this subset of invention polymers, diacid is included.

This subset of invention polymers can be described as having at least one A-moiety in which BA₂ is R.

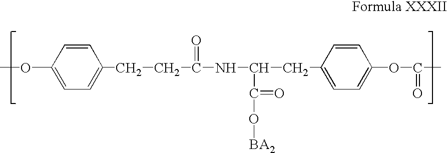

and at least one B-moiety

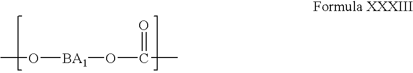

and at least one D-moiety

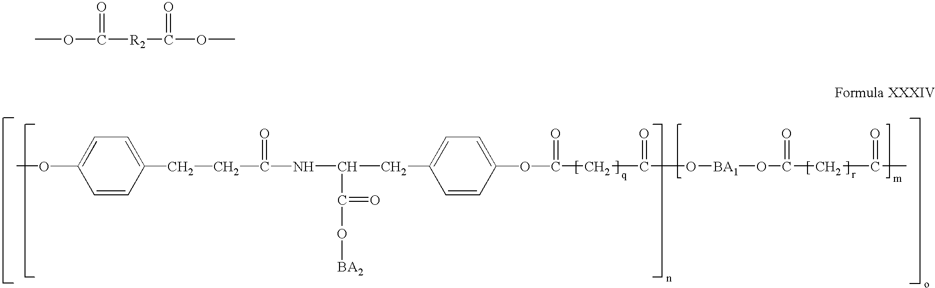

Formula XXXV, below, depicts an embodiment with the A-moieties, B-moieties, and D-moieties are combined.

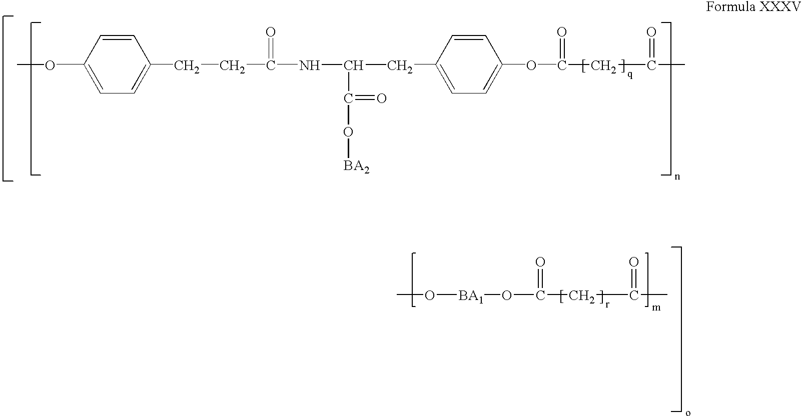

| | | | |
|---|---|---|---|
| m is | 0.005 to 0.995 | or | 0.01 to 0.99 |
| n is | 0.005 to 0.995 | or | 0.01 to 0.99 |
| o is | 4 to 3000 | or | 10 to 1500 |
| q is | 0.005 to 0.995 | or | 0.01 to 0.99 |
| r is | 0.005 to 0.995 | or | 0.01 to 0.99 |

The polymer represented by Formula XXXV is similar to Formula XII, above, except an aliphatic diacid is used instead of phosgene. This creates two important differences from Formula XII. The first is that hazardous phosgene is not required. The synthesis can be done with either diacid chlorides, using acid catalyzed condensation of the diacid, or carbodiimide coupling of the diol and diacid. These relatively safe processes can be done in-house. The second main difference is that this is a polyester polymer. The individual ester links are similar in reactivity to those in POLYACTIVE. Polyesters tend to be more crystalline than polycarbonates. But the pendant R group, and general complexity of the desamino tyrosyl-tyrosine dipeptide, may make this polymer amorphous. Consequently, its solvent solubility and degradation behavior will likely differ from POLYACTIVE's. Suitable diacids are oxalic acid, malonic acid, succinic acid, glutaric acid, adipic acid, pimelic acid, suberic acid, azelaic acid, and sebacic acid. Sebacic, adipic, and succinic acids are especially preferred. The polymerization can be carried out in two-step fashion. As the PEG has lower reactivity, it would be reacted first with a stoichiometric amount of diacid. This may be done by carbodiimide coupling. Useful solvents are methylene chloride or chloroform and appropriate carbodiimides are cyclohexylcarbodiimide or -diisopropylcarbodiimide. A stoichiometric to 100% excess of carbodiimide to molar quantity of ester linkages targeted would be added. A suitable catalyst is dimethylaminopyridinium-p-toluenesulfonate present in a molar ratio to carbodiimide of 1/100 to 1/5. After the PEG and diacid are allowed to react for a time to build the molecular weight of the soft segment, then desaminotyrosyl tyrosine alkyl ester with a stoichiometric amount of diacid would be added to build the hard segment. An additional amount of carbodiimide would also be required. An alternative scheme would be to have all of the diacid required for the synthesis present initially. PEG, carbodiimide, and catalyst would be added and allowed to react. As there is an excess of diacid, the reaction will only proceed to a certain extent. Then, the desaminotyrosyl tyrosine alkyl ester is added as a chain extender to build the final molecular weight and form the hard segment. Both schemes would create hard blocks of tyrosine-dipeptide/diacid and soft blocks of PEG/diacid.

Poly(desamino tyrosine tyrosyl hexyl ester succinate) has the structure below in Formula XXXVI.

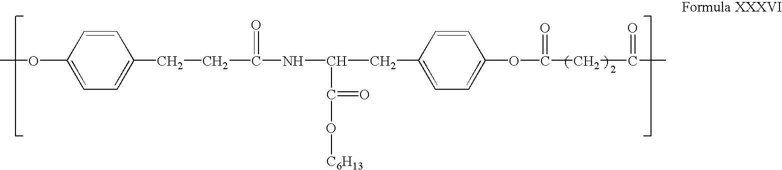

Unlike the tyrosine-derived polycarbonates, such as shown in Formula XII, this polymer is a poly(ester amide). A bioactive tyrosine ester with PEG in the backbone is shown below in Formula XXXVII.

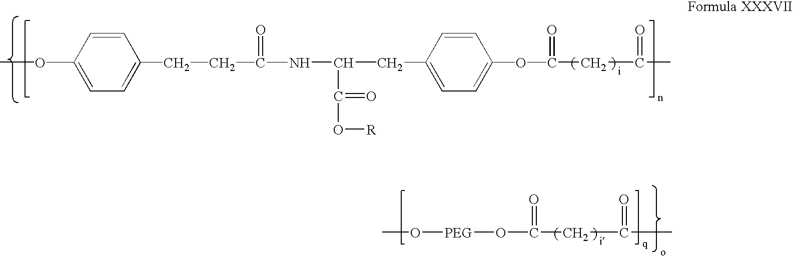

| | | | |
|---|---|---|---|
| n is | 0.05 to 0.99 | or | 0.10 to 0.90 |
| q is | 0.02 to 0.85 | or | 0.05 to 0.50 |
| o is | 5 to 2000 | or | 10 to 1200 |
| i is | 0 to 20 | or | 1 to 8 |
| i′ is | 0 to 20 | or | 1 to 8 |

In the invention polymer shown below in Formula XXXVIII, PEG replaces the R-group.

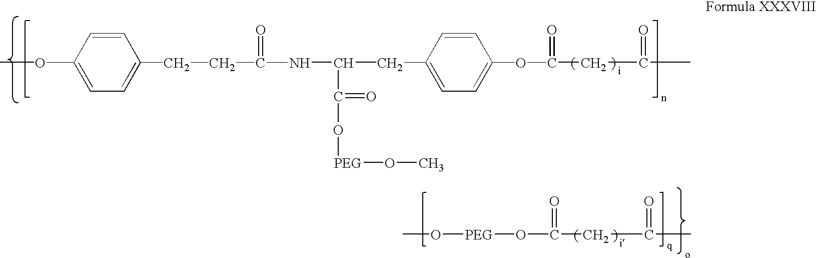

| | | | |
|---|---|---|---|
| n is | 0.02 to 0.95 | or | 0.1 to 0.80 |
| q is | 0.05 to 0.98 | or | 0.20 to 0.90 |
| o is | 1 to 1500 | or | 4 to 1000 |
| i is | 0 to 20 | or | 1 to 8 |
| i′ is | 0 to 20 | or | 1 to 8 |

An embodiment with two A-moieties, two B-moieties, and two D-moieties is shown below in Formula XL. BA₂ in the first A-moiety is PEG; BA₂ in the second A-moiety is HPSS. Similarly, BA₁ in the first B-moiety is HPSS; BA₁ in the second B-moiety is PVP. Finally, the R group for the first D-moiety is C₃H₆; the R group for the second D-moiety is shown below in Formula XXXIX

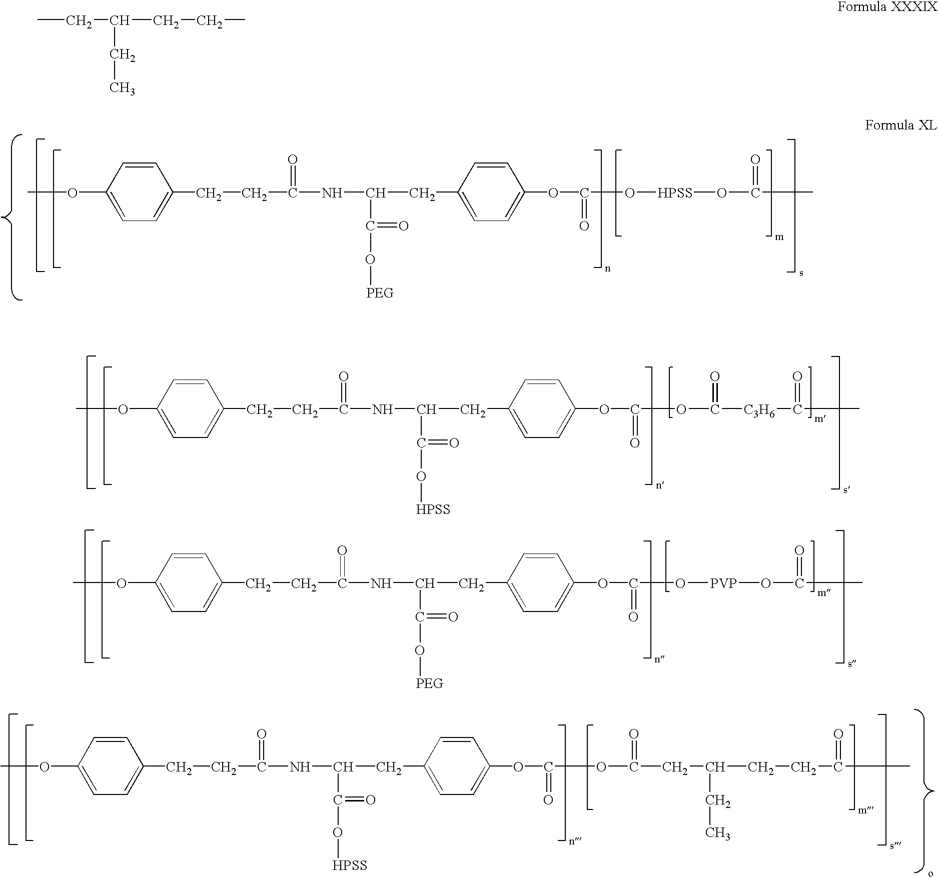

| | | | |
|---|---|---|---|
| m is | 0.005 to 0.90 | or | 0.01 to 0.75 |
| m′ is | 0.01 to 0.99 | or | 0.05 to 0.90 |
| m″ is | 0.005 to 0.90 | or | 0.01 to 0.75 |
| m′′′ is | 0.01 to 0.99 | or | 0.05 to 0.90 |
| n is | 0.01 to 0.95 | or | 0.05 to 0.75 |
| n′ is | 0.005 to 0.90 | or | 0.01 to 0.75 |
| n″ is | 0.01 to 0.95 | or | 0.05 to 0.75 |
| n′′′ is | 0.005 to 0.90 | or | 0.01 to 0.75 |
| s is | 0 to 0.80 | or | 0.05 to 0.50 |
| s′ is | 0 to 0.95 | or | 0.05 to 0.75 |
| s″ is | 0 to 0.80 | or | 0.05 to 0.50 |
| s′′′ is | 0 to 0.95 | or | 0.05 to 0.75 |
| o is | 1 to 2000 | or | 10 to 1000 |

Poly(Imino Carbonate) Tyrosine-Bioactive-Moiety-Copolymer Backbone

Another tyrosine-derived family of invention polymers that can be described are the polyiminocarbonates, shown below in Formula XLIII, which are imine analogs of polycarbonates. M₁ and M₂ are oxygen. M₃ and M₄ are NH.

This subset of invention polymers can be described as having at least one A-moiety

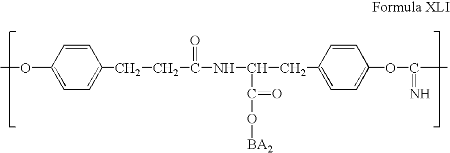

and at least one B-moiety

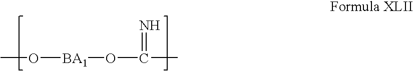

This embodiment is depicted below in Formula XLIII.

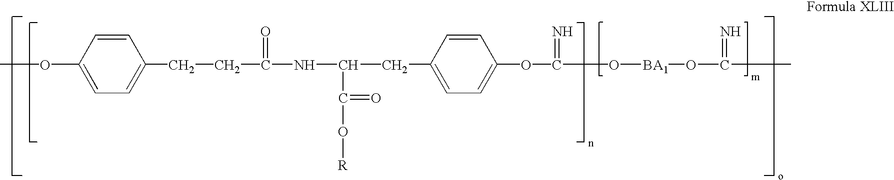

| | | | |
|---|---|---|---|
| m is | 0.005 to 0.99 | or | 0.05 to 0.95 |
| n is | 0.04 to 0.98 | or | 0.10 to 0.80 |
| o is | 2 to 4000 | or | 10 to 2000 |

Incorporating PEG or other bioactive moiety into the backbone yields another type of biobeneficial polymer, shown below as Formula XLIV.

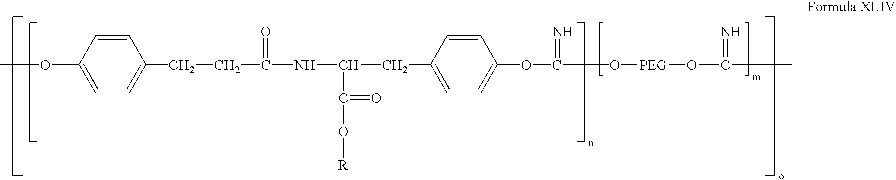

| | | | |
|---|---|---|---|
| m is | 0.02 to 0.96 | or | 0.05 to 0.75 |
| n is | 0.04 to 0.98 | or | 0.10 to 0.80 |
| o is | 2 to 2000 | or | 10 to 1000 |

Compared to tyrosine carbonates, such as shown above in Formula XII, the tyrosine imino carbonates, such as shown above in Formula XLIV are stronger but stiffer. They are also less stable towards hydrolysis, so they have a faster degradation rate in vivo.

Poly(Imino Tyrosine) Pendant PEG Groups

Poly(imino tyrosine) polymers with pendant bioactive moiety groups are shown below as Formula XLV. These polymers have a structure similar to the tyrosine carbonate embodiments, such as shown above in Formula XII. (it is an iminocarbonate).

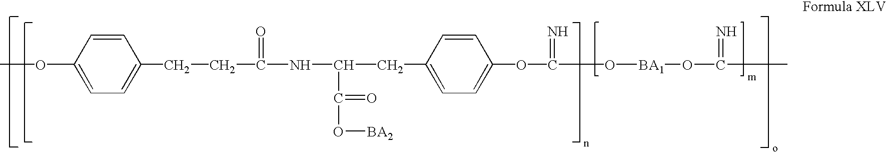

| | | | |
|---|---|---|---|
| m is | 0.005 to 0.99 | or | 0.05 to 0.95 |
| n is | 0.005 to 0.99 | or | 0.05 to 0.95 |
| o is | 2 to 4000 | or | 10 to 2000 |

When the bioactive moiety group is selected to be PEG the polymer has Formula XLVI, shown below.

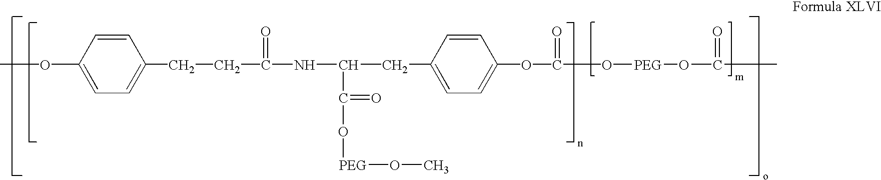

| | | | |
|---|---|---|---|
| m is | 0.02 to 0.96 | or | 0.05 to 0.75 |
| n is | 0.02 to 0.96 | or | 0.05 to 0.75 |
| o is | 2 to 2000 | or | 10 to 1000 |

Non-fouling moieties additionally include poly(propylene glycol), PLURONIC™ surfactants, poly(tetramethylene glycol), hydroxy functional poly(vinyl pyrrolidone), dextran, dextrin, sodium hyaluronate, and poly(2-hydroxyethyl methacrylate). A caveat is that the maximum molecular weight of this component should be low enough that this component is small enough to be released through the kidneys. In this respect, 40,000 daltons is the maximum molecular weight for some embodiments. In other embodiments, 20,000 is the maximum molecular weight.

Other bioactive moieties include (polyethylene glycol (PEG), poly(propylene glycol), poly(tetramethylene glycol), dihydroxy polyvinylpyrrolidone (PVP), dihydroxy poly(styrene sulfonate) (HPSS), poly(2-hydroxyethyl methacrylate), poly(3-hydroxypropyl methacrylates), poly(3-hydroxypropyl methacrylamide), poly(alkoxy methacrylates), poly(alkoxyacrylates), polyarginine peptides (PAP), such as R7, phosphoryl choline (PC), dextran, dextrin, sulfonated dextran, dermatan sulfate, heparin (HEP), chondroitan sulfate, glycosaminoglycans, chitosan, sodium hyaluronate or hyaluronic acid (HA).

In addition to being useful as non-fouling coatings, these polymers, due to their expected tunable hydration properties, may also be used for the delivery of proteins, peptides, and other biological molecules. These polymers may be coated onto a bare metal stent or they may be coated on top of a drug eluting coating already present on said stent. Conventional therapeutic agents, small hydrophobic drugs for example, may also be added to these bioabsorbable, non-fouling polymers making them bioabsorbable, drug eluting, coatings.

Various invention polymer embodiments can be branched or can be cross-linked, partially cross-linked, or not cross-linked, as desired. In some instances, cross-linking occurs through functional groups pendant from the polymer backbone. For instance, in some embodiments esters or amides in the backbone can serve as the cross-linking site. Those of ordinary skill in the art will recognize that other ways of achieving cross-links between polymer chains function with invention copolymers. For example, to UV crosslink the polymers, some embodiments have UV polymerizable groups in the monomers. Such groups are typically unsaturated diacids such as maleic or fumaric acid, unsaturated diols, acrylates or methacrylates. One general scheme would include replacing all or some of the diacid groups with maleic acid, fumaric acid, or other unsaturated diacid. Another scheme would place an acrylate, methacrylate, or cinnamate pendant on the R group of the desaminotyrosyl tyrosine moiety (A moiety). This gives rise to another class of polymers.

Some embodiments comprise invention polymers coated onto a medical device containing or constructed from a polymer, a medical device containing or constructed from a metal, or a bare medical device, or invention polymers coated on top of a drug coating already present on a medical device. Alternatively, some embodiments comprise invention polymers disposed between a medical device and a drug coating. Also, some embodiments comprise invention polymers composing polymer-based medical devices or invention polymers composing medical device substrates (implantable or not). Some invention embodiments comprise medical devices not made from polymer-containing or -constructed stents. Some invention embodiments comprise stents not made from metal-containing or constructed stents.

In some embodiments, invention polymers serve as the base material for coatings on medical devices. In some embodiments, coatings may contain a primer layer composed of an invention polymer or composed of a type-two polymer, as described below. Some embodiments exclude a primer layer.

Some embodiments add conventional drugs, such as small, hydrophobic drugs, to invention polymers (as discussed in any of the embodiments, above), making them biodegradable drug systems. Some embodiments graft on conventional drugs or mix conventional drugs with invention polymers. Invention polymers can be coated as blends with a variety of biobeneficial polymers. Moreover, they can serve as base or topcoat layers for biobeneficial polymer layers.

The selected drug can inhibit vascular, smooth muscle cell activity. More specifically, the drug activity can aim at inhibiting abnormal or inappropriate migration or proliferation of smooth muscle cells to prevent, inhibit, reduce, or treat restenosis. The drug can also include any substance capable of exerting a therapeutic or prophylactic effect in the practice of the present invention.

These agents can have anti-proliferative or anti-inflammmatory properties or can have other properties such as antineoplastic, antiplatelet, anti-coagulant, anti-fibrin, antithrombonic, antimitotic, antibiotic, antiallergic, antioxidant as well as cystostatic agents. Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which bind to complementary DNA to inhibit transcription, and ribozymes. Some other examples of other bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. Examples of anti-proliferative agents include rapamycin and its functional or structural derivatives, 40-O-(2-hydroxy)ethyl-rapamycin (everolimus), and its functional or structural derivatives, paclitaxel and its functional and structural derivatives. Examples of rapamycin derivatives include methyl rapamycin (ABT-578), 40-O-(3-hydroxy)propyl-rapamycin, 40-O-[2-(2-hydroxy)ethoxy]ethyl-rapamycin, and 40-O-tetrazolerapamycin. Examples of paclitaxel derivatives include docetaxel. Examples of antineoplastics and/or antimitotics include methotrexate, azathioprine, vincristine, vinblastine, fluorouracil, doxorubicin hydrochloride (e.g. Adriamycino from Pharmacia & Upjohn, Peapack N.J.), and mitomycin (e.g. Mutamycine from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, thrombin inhibitors such as Angiomax a (Biogen, Inc., Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO), estradiol, anticancer agents, dietary supplements such as various vitamins, and a combination thereof. Examples of antiinflammatory agents including steroidal and non-steroidal anti-inflammatory agents include tacrolimus, dexamethasone, clobetasol, combinations thereof. Examples of such cytostatic substance include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.). An example of an antiallergic agent is permirolast potassium. Other therapeutic substances or agents which may be appropriate include alpha-interferon, bioactive RGD, and genetically engineered epithelial cells. The foregoing substances can also be used in the form of prodrugs or co-drugs thereof. The foregoing substances are listed by way of example and are not meant to be limiting. Other active agents which are currently available or that may be developed in the future are equally applicable.

The dosage or concentration of the bioactive agent required to produce a favorable therapeutic effect should be less than the level at which the bioactive agent produces toxic effects and greater than the level at which non-therapeutic results are obtained. The dosage or concentration of the bioactive agent required can depend upon factors such as the particular circumstances of the patient; the nature of the tissues being delivered to; the nature of the therapy desired; the time over which the ingredient administered resides at the vascular site; and if other active agents are employed, the nature and type of the substance or combination of substances. Therapeutic effective dosages can be determined empirically, for example by infusing vessels from suitable animal model systems and using immunohistochemical, fluorescent or electron microscopy methods to detect the agent and its effects, or by conducting suitable in vitro studies. Standard pharmacological test procedures to determine dosages are understood by one of ordinary skill in the art.

Individual embodiments exist in which the drug is selected to specifically exclude any one of or any combination of the drugs or drug families described above.

Some invention embodiments comprise a drug or drug combination, and some require a drug or combination of drugs. Of the drugs specifically listed above, some invention embodiments exclude a single or any combination of these drugs.

These blends could also be formulated to modulate or tune the release rate of drugs from coatings, reservoirs, or particles composed of these blends and drugs or therapeutic agents. Blends with other polymers (called type-two polymers for the purpose of this disclosure) can be formulated to modulate the mechanical properties of invention polymers. Preferably, the polymers blended with the invention polymers would be biodegradable as that leads to a uniformly biodegradable system. However, they may also be durable as the blend can have other useful properties. Predictable properties may be obtained if the type-two polymers are miscible with the invention polymers. However, as the invention polymers span a range of polarities and solubility parameters, the range of type two polymers that can be miscible is also large. Furthermore, microstructural phase separation, as occurs in ABS for example, of the invention polymer and type-two polymer can also be desired in some instances as it can lead to useful mechanical properties.

Type-two polymers could be blended into invention polymers to modify mechanical properties, biological properties, degradation rates, or drug release properties.

In some embodiments, invention mixtures comprise an invention polymer and a type-two polymer. The following polymer families can be the source of type-two polymers in invention polymer mixtures: ABS resins; acrylic polymers and copolymers; acrylonitrile-styrene copolymers; alkyd resins; biomolecules; cellulose ethers; celluloses; copoly(ether-esters) (e.g. PEO/PLA); copolymers of vinyl monomers with each other and olefins; cyanoacrylates; epoxy resins; ethylene-a-olefin copolymers; ethylene-methyl methacrylate copolymers; ethylene-vinyl acetate copolymers; poly(amino acids); poly(anhydrides); poly(ester amides); poly(imino carbonates); poly(orthoesters); poly(ester amides); poly(tyrosine arylates); poly(tyrosine derive carbonates); polyalkylene oxalates; polyamides; polyanhydrides; polycarbonates; polyesters; polyethers; polyimides; polyolefins; polyorthoester; polyoxymethylenes; polyphosphazenes; polyphosphoester; polyphosphoester urethane; polyurethanes; polyvinyl aromatics; polyvinyl esters; polyvinyl ethers; polyvinyl ketones; polyvinylidene fluoride; silicones; starches; vinyl halide polymers and copolymers; other biobeneficial polymers; and their combinations. Some invention embodiments are defined such that a type-two polymer excludes any one or any combination of polymers selected from the families listed above.

The following polymers can be used as type-two polymers in invention polymer mixtures: poly(butyl methacrylates); poly(alkoxy acrylates); poly(alkoxy methacrylates); carboxymethyl cellulose; cellophane; cellulose; methyl cellulose; ethyl cellulose; cellulose acetate; hydroxyethyl cellulose; hydroxypropyl cellulose; cellulose acetate butyrate; cellulose butyrate; cellulose nitrate; cellulose propionate; collagen; ethylene vinyl alcohol copolymer; poly(vinyl alcohol); fibrin; fibrinogen; hyaluronic acid; Nylon 66; poly(L-lactide); poly(L-lactic acid), poly(D-lactide), poly(D-lactic acid), poly(D,L-lactic acid), poly(glycolide); poly(L-lactide-coglycolide); poly(D,L-lactide-co-glycolide); poly(caprolactone), poly(L-lactide-co-caprolactone); poly(D,L-lactide-co-caprolactone); polydioxanone; poly(trimethylene carbonate); poly(3-hydroxy valerate); poly(3-hydroxybutyrate); poly(4-hydroxybutyrate); poly(D,L-lactic acid); poly(D,L-lactide); poly(D,L-lactide-co-glycolide); poly(D,L-lactide-co-L-lactide); poly(dioxanone); poly(glycolic acid); poly(glycolic acid-co-trimethylene carbonate); poly(glycolide); poly(hydroxybutyrate); poly(hydroxybutyrate-co-hydroxyvalerate); poly(hydroxybutyrate-co-valerate); poly(hydroxyvalerate); poly(iminocarbonate); poly(lactide-co-glycolide); poly(L-lactic acid); poly(L-lactide); poly(trimethylene carbonate); polyacrylonitrile; polycaprolactam; polycaprolactone; polydioxanone; polyisobutylene; polystyrene; styrene-ethylene/butylene-styrene triblock copolymers; styrene-isobutylene-styrene triblock copolymers; poly(vinylidene fluoride-co-chlorotrifluoroethylene); poly(vinylidene fluoride-co-hexafluoropropylene); poly(vinyl fluoride); polyvinyl acetate; PEG; POLYACTIVE; and their combinations. Some invention embodiments are defined such that a type-two polymer excludes any one or any combination of the polymers listed above.

Some invention embodiments comprise, and some invention embodiments require, a type-two polymer used along with invention polymers. Some invention embodiments comprise and some invention embodiments require combining at least two type-two polymers with invention polymers. Of the type-two polymers disclosed above, some invention embodiments exclude a single or any combination of type-two polymers.

In some embodiments in which invention polymers are used with type-two polymers, the invention polymers are mixed or blended with the type-two polymers. For example, some embodiments comprise invention polymers physically blended type-two polymers.

Some embodiments comprise invention polymers combined with other polymers in multilayer arrangements. For example, an invention polymer could under- or over-lay another polymer such as a polymer coated on a device, a medical device, an implantable medical device, or a stent. The invention polymer can be used neat in this regard, or it can first be mixed with a separate invention polymer or a type-two polymer before layering. In some embodiments, invention polymers do not underlay another polymer; in other embodiments, invention polymers must overlay another polymer.

Examples of implantable devices useful in the present invention include self-expandable stents, balloon-expandable stents, stent-grafts, grafts (e.g., aortic grafts), vascular grafts, artificial heart valves, cerebrospinal fluid-shunts, pacemaker electrodes, guidewires, closure devices for patent foramen ovale, ventricular assist devices, artificial hearts, cardiopulmonary by-pass circuits, blood oxygenators, and endocardial leads (e.g., FINELINE and ENDOTAK, available from Guidant Corporation, Santa Clara, Calif.). The underlying structure of the device can be of virtually any design. The device can comprise a metallic material or an alloy such as, but not limited to, cobalt chromium alloy (ELGILOY), stainless steel (316L), high nitrogen stainless steel, e.g., BIODUR 108, cobalt chrome alloy L-605, “MP35N,” “MP20N,” ELASTINITE (Nitinol), tantalum, nickel-titanium alloy, platinum-iridium alloy, gold, magnesium, or combinations thereof. “MP35N” and “MP20N” are trade names for alloys of cobalt, nickel, chromium, and molybdenum available from Standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum. Devices made from bioabsorbable or biostable polymers could also be used with the embodiments of the present invention. A hemocompatible or antithrombotic surface has the potential to reduce the problem of delayed thrombosis. A biobeneficial surface of sufficient duration in vivo has the potential to reduce the foreign body response and chronic inflammation.

Some invention embodiments define the genre of medical devices to exclude at least one of self-expandable stents, balloon-expandable stents, stent-grafts, grafts (e.g., aortic grafts), vascular grafts, artificial heart valves, cerebrospinal fluid shunts, pacemaker electrodes, guidewires, ventricular assist devices, artificial hearts, cardiopulmonary by-pass circuits, blood oxygenators, or endocardial leads.

A coating for an implantable medical device, such as a stent, according to embodiments of the present invention, can be a multi-layer structure that can include any one or any combination of the following four layers:

(a) a primer layer;

(b) a drug-polymer layer (also referred to as “reservoir” or “reservoir layer”) or a polymer-free drug layer; and

(c) a topcoat layer, which is likewise drug containing or drug free.

(d) a finishing layer, for biocompatibility possessing biobeneficial properties.

In some embodiments, forming each medical device coating layer comprises dissolving the polymer or a polymer blend in a solvent or a solvent mixture, and applying the solution onto the medical device (such as by spraying the medical device with the solution or by dipping the medical device into the solution). After applying the solution onto the medical device, the coating dries by solvent evaporation. Drying at elevated temperatures accelerates the process.

Combining the drug with the polymer solution, as described above, provides for incorporating the drug into the reservoir layer. Alternatively, dissolving the drug in a suitable solvent or solvent mixture and applying the drug solution to the medical device provides for a substantially polymer-free drug layer.

Instead of introducing the drug as a solution, the drug can be introduced as a colloid, such as a suspension in a solvent. Dispersing the drug in the solvent uses conventional techniques. Depending on a variety of factors, e.g., the nature of the drug, those having ordinary skill in the art can select the solvent for the suspension, as well as the quantity of the dispersed drug. Some embodiments mix these suspensions with a polymer solution and apply the mixture onto the device, as described above. Alternatively, some embodiments apply the drug suspension to the device without mixing it with the polymer solution.

The drug-polymer layer can be applied directly onto at least a part of the medical device surface to serve as a reservoir for at least one active agent or a drug. The drug containing layer may only be applied ablumenally, lumenally, to strut sidewalls, or to any combination of the three. The optional primer layer can be applied between the device and the reservoir to improve polymer adhesion to the medical device. Some embodiments apply the topcoat layer over at least a portion of the reservoir layer, and the topcoat layer serves as a rate limiting membrane that helps to control the rate of release of the drug.

Some drug releasing processes include at least two steps. First, the topcoat polymer absorbs the drug at the drug-polymer-topcoat interface. Next, the drug diffuses through the topcoat using the free volume of the polymer molecules as diffusion pathways. Next, the drug arrives to the outer surface of the topcoat, and desorbs into the surrounding tissue or blood stream.

### EXAMPLE SYNTHESIS

### Example 1

### Synthesis of Polymer Formula XIV

Synthesis of carbobenzoxy protected L-tyrosine: To a 1000 ml flask equipped with ice bath and magnetic stirrer is added methanol (250 ml), L-tyrosine (100 gm, 0.552 mole), triethylamine (84.6 ml, 0.607 mole), and benzyl chloroformate (94.2 gm, 0.552 mole). After stirring for 2 hours, the solution is poured into 2 liters of ice water and extracted with three, 500 ml portions of diethylether. The ethereal extracts are combined and washed with one 250 ml portion of 5% acetic acid buffer. After drying the ether phase over magnesium sulfate, the solvent is removed by rotary evaporation and the resulting carbobenzoxy protected tyrosine is dried in vacuum.

Synthesis of tyrosine methoxy-PEG5000 amide: To a 500 ml flask equipped with ice bath, argon inlet and magnetic stirrer is added tetrahydrofuran (200 ml), carbobenzoxy-L-tyrosine (2 gm, 6.35 mmole), methoxy-polyethyleneglycol-amine (MW 5000, available from Nektar, Huntsville, Ala.) (31.75 gm, 6.35 mmole), and hydroxyl-benzotriazole (0.946 gm, 7 mmol). After dissolution, dicyclohexylcarbodiimide (1.44 gm, 7 mmol) is added and the reaction stirred for 1 hour at 0° C. and then overnight at ambient temperature. Glacial acetic acid (0.21 gm, 3.5 mmol) is added and the solution is filtered to remove the dicyclohexylurea. After concentrating the solution by rotary evaporation, it is dissolved in 200 ml of methylene chloride and extracted with one 200 ml portion of 0.1 N aqueous HCl, and one 200 ml portion of 0.1N aqueous sodium carbonate. After drying over magnesium sulfate, the solvent is removed by rotary evaporation and the carbobenzoxy tyrosine mPEG amide dried in vacuum.

Hydrogenolysis of carbobenzoxy L-tyrosine mPEG amide: To a 500 ml flask equipped with argon inlet, vacuum line, and hydrogen gas inlet is added palladium (2 gm, 0.019 moles) and vacuum applied. After purging with argon, ethanol (200 ml) is added and hydrogen bubbled through the solution for 30 minutes. Carbobenzoxy tyrosine mPEG amide (20 gm, 3.78 mmol) is added under argon, dissolved, and the solution stirred with a steady bubbling of hydrogen for 12 hours. The palladium is removed by filtration and the ethanol solution added dropwise to 1 liter of ethyl acetate. The tyrosine-mPEG-amide is collected and dried in vacuum.

Synthesis of desaminotyrosyl tyrosine mPEG amide: To a 100 ml flask equipped with magnetic stirrer, argon purge, and ice bath is added tetrahydrofuran (50 ml), desaminotyrosine (0.29 gm, 1.94 mmole), tyrosine-mPEG-amide (10 gm, 1.94 mmol), and hydroxyl-benzotriazole (0.284 gm, 2.1 mmol). After dissolution, dicylohexylcarbodiimide (0.433 gm, 2.1 mmol) is added and the solution stirred at °0 C for one hour and then overnight at ambient temperature. Glacial acetic acid is added (50 mg, 0.83 mmol), the dicyclohexylurea removed by filtration, and the solution concentrated by rotary evaporation. It is dissolved in 50 ml of methylene chloride and extracted with one 50 ml portion of 0.1 N aqueous HCl and one 50 ml portion of 0.1N aqueous sodium carbonate. After drying over magnesium sulfate, the methylene chloride is removed in vacuum yielding desaminotyrosyl tyrosine mPEG amide.

Synthesis of co-poly-{[desaminotyrosyl tyrosine mPEG amide]_{0.0256}-[desaminotyrosyl tyrosine ethyl ester]0.974}: To a 1000 ml round bottom flask equipped with mechanical stirrer and argon inlet is added desaminotyrosyl tyrosine ethyl ester (27.3 gm, 0.071 mole), desaminotyrosyl tyrosine mPEG amide (10 gm, 1.87 mmole), anhydrous methylene chloride (200 ml), and anhydrous pyridine (21.62 gm, 0.273 mole). After dissolution, and at ambient temperature, phosgene (9.01 gm, 0.0911 mole phosgene) as a 20% solution in toluene is added slowly with stirring. After stirring another two hours, tetrahydrofuran (600 ml) is added and the polymer precipitated by slow addition to 5 liters of a 75/25 (w/w) blend of hexane/ethyl acetate. After isolating the polymer, it is redissolved in THF (400 ml) and precipitated in deionized water (4000 ml). After a final dissolution in methylene chloride (800 ml), the solution is filtered through a dry disc apparatus (Horizon Technology, Atkinson, N.H.) with a Teflon™ filter to remove water, the solvent removed by rotary evaporation, and the polymer dried in vacuum. This yields a polymer of formula XIV with a pendant mPEG group of 5000 Dalton molecular weight, and a weight fraction of mPEG in the polymer of 25%.

### Example 2

### Synthesis of Polymer Formula XXIII

To a 1000 ml round bottom flask equipped with mechanical stirrer and argon inlet is added methylene chloride (200 ml), desaminotyrosyl tyrosine ethyl ester (25 gm, 0.07 mol), anhydrous PEG 300 (15.3 gm, 0.051 mol), and pyridine (41.5 gm, 0.525 mol). After dissolution, phosgene (17.31 gm, 0.175 moles) is added dropwise as a 20% solution in toluene at ambient temperature over 60 minutes. Anhydrous 1,4-butanediol (1.71 gm, 0.019 moles) is added, and the solution stirred for another 60 minutes. It is diluted with THF (700 ml) and the polymer precipitated by slow addition to 5 liters of a 75/25 (w/w) blend of hexane/ethyl acetate. After isolation, the polymer is redissolved in THF (400 ml) and precipitated into deionized water (4 liters). After a final dissolution in methylene chloride (800 ml), the solution is filtered through a dry disc apparatus (Horizon Technology, Atkinson, N.H.) with a Teflon™ filter to remove water, the solvent removed by rotary evaporation, and the polymer dried in vacuum. This yields a polymer of formula XXIII with hard blocks, and PEG containing soft blocks, where the PEG 300 moieties are in the polymer backbone. The weight fraction of PEG in the polymer is 33%.

### Example 3

### Synthesis of Polymer Formula XXXVI

To a 1000 ml round bottom flask equipped with mechanical stirrer and argon purge is added PEG 600 (25 gm, 0.0417 mol), adipic acid (12.23 gm, 0.0838 mol), desaminotyrosyl tyrosine butyl ester (20.25 gm, 0.0421 mol) and dimethylaminopyridinium p-toluenesulfonate (9.858 gm, 0.0335 mol). Next methylene chloride (500 ml) is added and the reactants dissolved. Diisopropylcarbodiimide (42.3 gm, 0.335 moles) is added and the solution stirred under argon at ambient temperature for 24 hours. The reaction mixture filtered to remove the diisopropylurea and slowly added to diethyl ether (5000 ml) with stirring to precipitate the polymer. The polymer is redissolved in methylene chloride (500 ml) and further purified by slow addition to diethyl ether (5000 ml), after which it is collected and dried in vacuum. This yields a poly(ester amide) polymer of formula XXXVI containing the PEG 600 moieties in the polymer backbone. with a weight fraction of PEG in the polymer of 50%.

### Example 4

### Coating a Stent with the Composition of Example 1

A composition can be prepared by mixing the following components:

about 2.0% (w/w) of the polymer of Example 1; and

about 0.2% (w/w) of paclitaxel

(c) the balance a 50/50 (w/w) blend of chloroform and 1,1,2-trichloroethane.

The composition can be applied onto the surface of bare 12 mm small VISION™ stent (Guidant Corp.). The coating can be sprayed and dried to form a drug reservoir layer. A spray coater can be used having a 0.014 round nozzle maintained at ambient temperature with a feed pressure 2.5 psi (0.17 atm) and an atomization pressure of about 15 psi (1.02 atm). About 20 μg of the coating can be applied at per one spray pass. About 180 μg of wet coating can be applied, and the stent can be dried for about 10 seconds in a flowing air stream at about 50° C. between the spray passes. The stents can be baked at about 50° C. for about one hour, yielding a drug reservoir layer composed of approximately 150 μg of the polymer of Example 1 and about 14 μg of paclitaxel.

### Example 5

### Coating a Stent with the Composition of Example 3

A first composition can be prepared by mixing the following components:

(a) about 2.0% (w/w) of poly(butyl methacrylate); and

(b) the balance a 50/50 (w/w) blend of acetone and cyclohexanone.

The first composition can be applied onto the surface of bare 12 mm small VISION™ stent (Guidant Corp.). The coating can be sprayed and dried to form a primer layer. A spray coater can be used having a 0.014 round nozzle maintained at ambient temperature with a feed pressure 2.5 psi (0.17 atm) and an atomization pressure of about 15 psi (1.02 atm). About 20 μg of the coating can be applied at per one spray pass. About 100 μg of wet coating can be applied, and the stent can be dried for about 10 seconds in a flowing air stream at about 50° C. between the spray passes. The stents can be baked at about 80° C. for about one hour, yielding a primer layer composed of approximately 80 μg of poly(butyl methacrylate).

A second composition can be prepared by mixing the following components:

(a) about 2.0% (w/w) of poly(vinylidene fluoride);

(b) about 1.0% (w/w) everolimus; and

(c) the balance a 50/50 (w/w) blend of acetone and cyclohexanone.

The second composition can be applied onto the dried primer layer to form the drug-polymer layer using the same spraying technique and equipment used for applying the primer layer. About 330 μg of wet coating can be applied followed by drying and baking at about 50° C. for about 2 hours, yielding a dry drug-polymer layer having solids content of about 300 μg, containing about 100 μg of everolimus.

A third composition can be prepared by mixing the following components:

(a) about 2.0% (w/w) the polymer of Example 3; and

(b) the balance a 50/50 (w/w) blend of chloroform and dimethylformamide.

The third composition can be applied onto the dried drug-polymer layer to form a biobeneficial finishing layer using the same spraying technique and equipment used for applying the primer and drug-polymer layers. About 110 μg of wet coating can be applied followed by drying and baking at about 50° C. for about 1 hour, yielding a dry biobeneficial finishing layer having solids content of about 100 μg.

While particular embodiments of the present invention have been shown and described, it will be obvious to those skilled in the art that changes and modifications can be made without departing from the embodiments of this invention in its broader aspects and, therefore, the appended claims are to encompass within their scope all such changes and modifications as fall within the true spirit and scope of the embodiments of this invention. Additionally, various embodiments have been described above. For convenience's sake, combinations of aspects (such as monomer type or gas flow rate) composing invention embodiments have been listed in such a way that one of ordinary skill in the art may read them exclusive of each other when they are not necessarily intended to be exclusive. But a recitation of an aspect for one embodiment is meant to disclose its use in all embodiments in which that aspect can be incorporated without undue experimentation. In like manner, a recitation of an aspect as composing part of an embodiment is a tacit recognition that a supplementary embodiment exists that specifically excludes that aspect.

Moreover, some embodiments recite ranges. When this is done, it is meant to disclose the ranges as a range, and to disclose each and every point within the range, including end points. For those embodiments that disclose a specific value or condition for an aspect, supplementary embodiments exist that are otherwise identical, but that specifically exclude the value or the conditions for the aspect.

What is claimed is:

1. A polymer comprising
an A-moiety with the formula 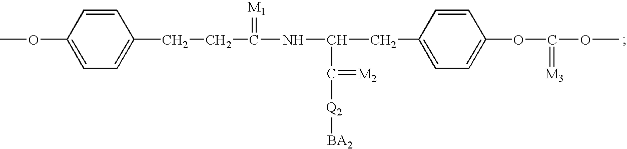
optionally, a B-moiety with the formula 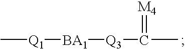
optionally, a C-type moiety, diol diradical, with the formula
—O—R₃—O—; and
optionally, a D-type moiety, diacid diradical, with the formula 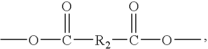
wherein
M₁-M₄ are independently selected from O, NH, CH₂, or S;
Q₁-Q₃ are independently selected from Group-15- or Group-16-containing moieties;
BA₁ and BA₂ are independently selected from an R-group or a bioactive moiety, wherein the R-group is a C₁-C₂₀, linear or branched, (un)substituted alkyl or aryl and provided that at least 0.001 or 0.01 mole percent of the total of BA₁ and BA₂ are selected from one or more bioactive moieties; and
wherein if the polymer contains none of B-moiety, C-moiety, and D-moiety, it comprises at least two different A-moieties.

2. The polymer of claim 1 wherein M₁-M₄ are independently selected from O and NH.

3. The polymer of claim 1 wherein Q₁-Q₃ are independently selected from N-, O-, S-, P-, and Se-containing moieties

4. The polymer of claim 2 wherein Q₁-Q₃ are independently selected from NH, NR⁴, or O wherein R⁴ is a C₁-C₂₀, linear or branched, (un)substituted alkyl or aryl.

5. The polymer of claim 1 wherein bioactive moieties are selected from PEG, PPG, poly(tetramethylene glycol), PVP, HPSS, PHEMA, poly(3-hydroxypropyl methacrylates), PHPMA, poly(alkoxy methacrylates), poly(alkoxy acrylates), PAP, R7, PC, dextran, dextrin, sulfonated dextran, dermatan sulfate, HEP, chondroitan sulfate, glycosaminoglycans, chitosan, sodium hyaluronate, HA, or any combination of these.

6. The polymer of claim 1 comprising at least one of the B-, C-, or D-moieties or a combination of these moieties.

7. The polymer of claim 1 wherein diol diradical comprises 1-12 carbon atoms and has at least two radicals derived from alcohol groups.

8. The polymer of claim 1 wherein diol is 1,4-butanediol.

9. The polymer of claim 1 wherein diacid diradical comprises 2-30 carbon atoms and has at least two radicals derived from carboxylic acid groups.

10. The polymer of claim 1 wherein diacids are selected from oxalic acid, malonic acid, succinic acid, glutaric acid, adipic acid, pimelic acid, suberic acid, azelaic acid, maleic acid, fumaric acid, and sebacic acid.

11. A polymer comprising
an A-moiety with the formula 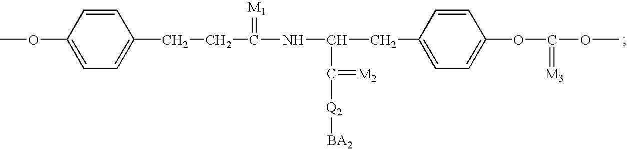
optionally, a B-moiety with the formula 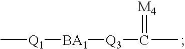
optionally, a C-type moiety, diol diradical, with the formula
—O—R₃—O—; and
optionally, a D-type moiety, diacid diradical, with the formula 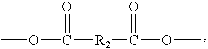
wherein
M₁-M₄ are independently selected from O and NH;
Q₁-Q₃ are selected from NH, NR⁴, or O wherein R⁴ is a C₁-C₂₀, linear or branched, (un)substituted alkyl or aryl;
BA₁ and BA₂ are independently selected from an R-group or a bioactive moiety, wherein the R-group is a C₁-C₂₀, linear or branched, (un)substituted alkyl or aryl and provided that at least 0.001 mole percent of the total of BA₁ and BA₂ are selected from one or more bioactive moieties selected from PEG, PPG, poly(tetramethylene glycol), PVP, HPSS, PHEMA, poly(3-hydroxypropyl methacrylates); PHPMA, poly(alkoxy methacrylates), poly(alkoxy acrylates), PAP, R7, PC, dextran, dextrin, sulfonated dextran, dermatan sulfate, HEP, chondroitan sulfate, glycosaminoglycans, chitosan, sodium hyaluronate, HA, or any combination of these;
diol diradicals comprise 1-12 carbon atoms and have at least two radicals derived from alcohol groups;
diacids are selected from oxalic acid, malonic acid, succinic acid, glutaric acid, adipic acid, pimelic acid, suberic acid, azelaic acid, maleic acid, fumaric acid, sebacic acid, or their mixtures; and
wherein if the polymer contains none of B-moiety, C-moiety, and D-moiety, it comprises at least two different A-moieties.

12. A polymer comprising
an A-moiety with the formula 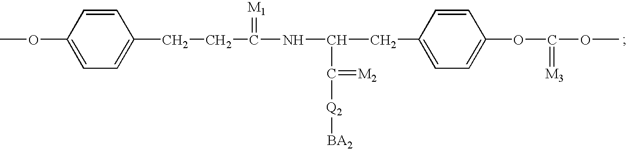
optionally, a B-moiety with the formula 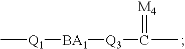
optionally, a C-type moiety, diol diradical, with the formula
—O—R₃—O—; and
optionally, a D-type moiety, diacid diradical, with the formula 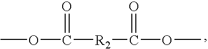
wherein
M₁-M₄ are independently selected from O and NH;
Q₁-Q₃ are selected from NH, NR⁴, or O wherein R⁴ is a C₁-C₂₀, linear or branched, (un)substituted alkyl or aryl;
BA₁ and BA₂ are independently selected from an R-group or a bioactive moiety, wherein the R-group is a C₁-C₂₀, linear or branched, (un)substituted alkyl or aryl and provided that at least 0.001 or 0.01 mole percent of the total of BA₁ and BA₂ are selected from PEG, PPG, poly(tetramethylene glycol), PVP, HPSS, PHEMA, poly(3-hydroxypropyl methacrylates), PHPMA, poly(alkoxy methacrylates), poly(alkoxy acrylates), PAP, R7, PC, dextran, dextrin, sulfonated dextran, dermatan sulfate, HEP, chondroitan sulfate, glycosaminoglycans, chitosan, sodium hyaluronate, HA, or any combination of these;
diols are 1,4-butanediol;
diacids are selected from oxalic acid, malonic acid, succinic acid, glutaric acid, adipic acid, pimelic acid, suberic acid, azelaic acid, maleic acid, fumaric acid, sebacic acid, or their mixtures; and
wherein if the polymer contains none of B-moiety, C-moiety, and D-moiety, it comprises at least two different A-moieties.

13. The polymer of claim 1 with one of the following formulas: 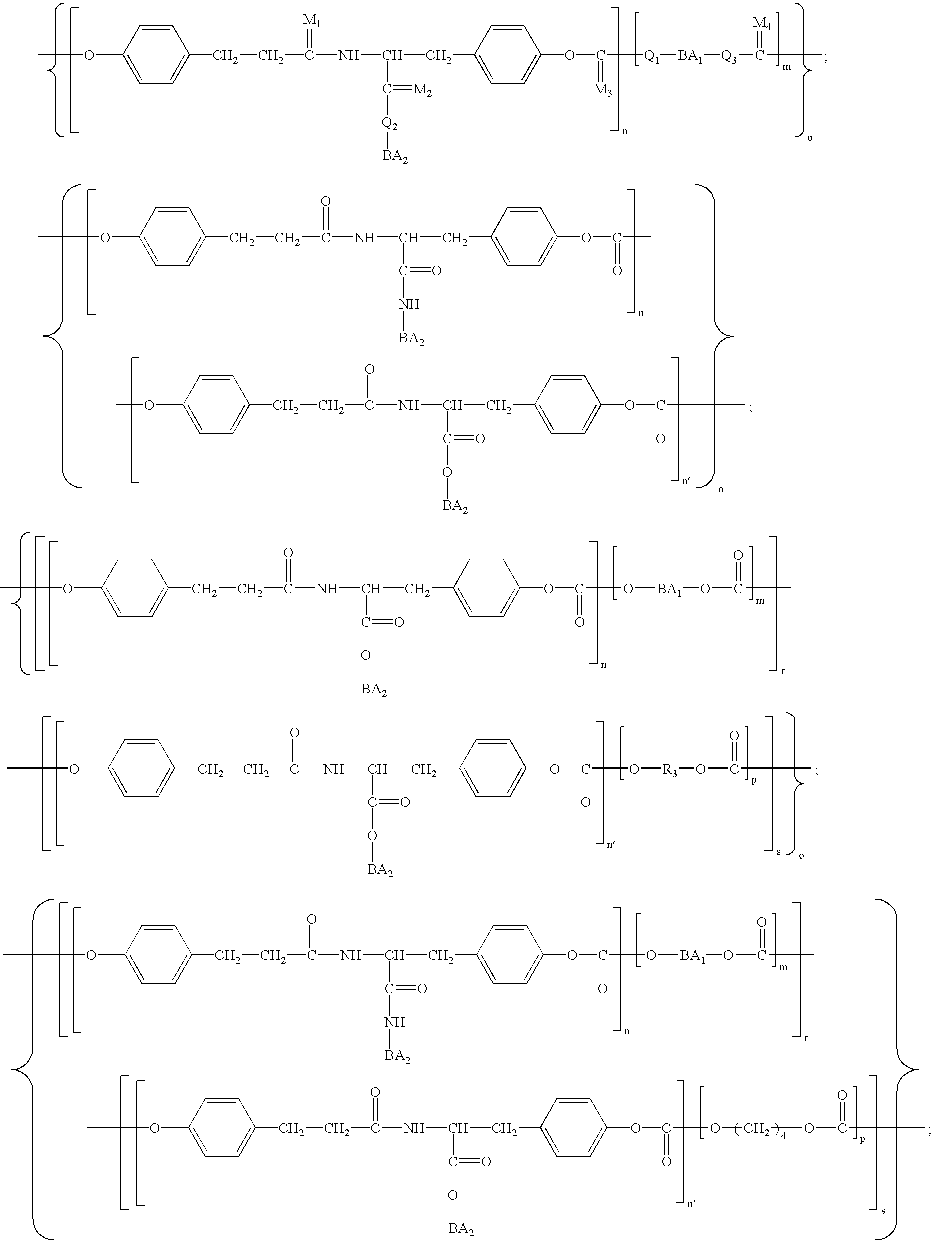 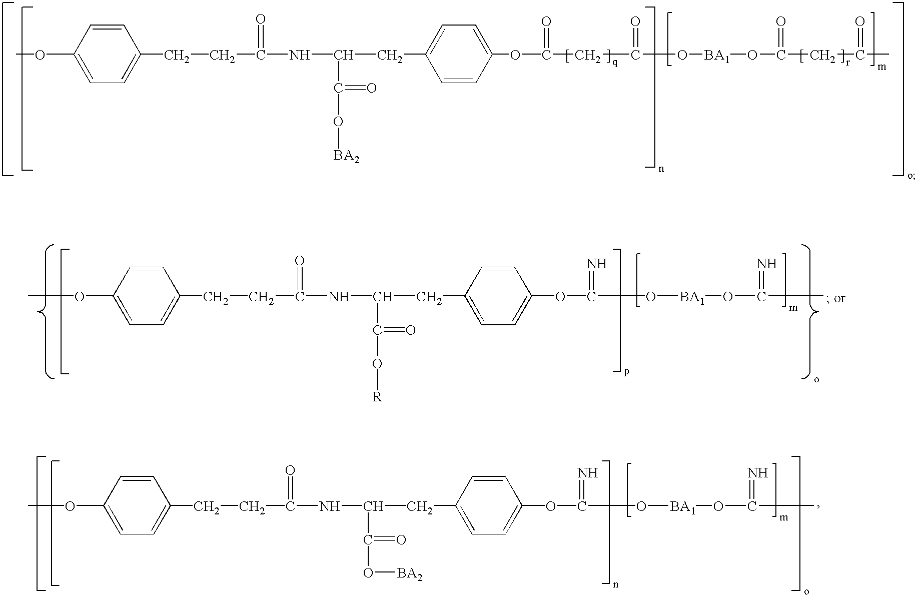
o is 4 to 3000
m is 0.005 to 0.995;
p is 0.1 to 0.99
i is 0 to 20;
i′ is 0 to 20;
n is 0.005 to 0.995;
s is 0.25 to 0.99; and
n′ is 0 to 0.99.

14. The polymer of claim 1 with one of the following formulas: 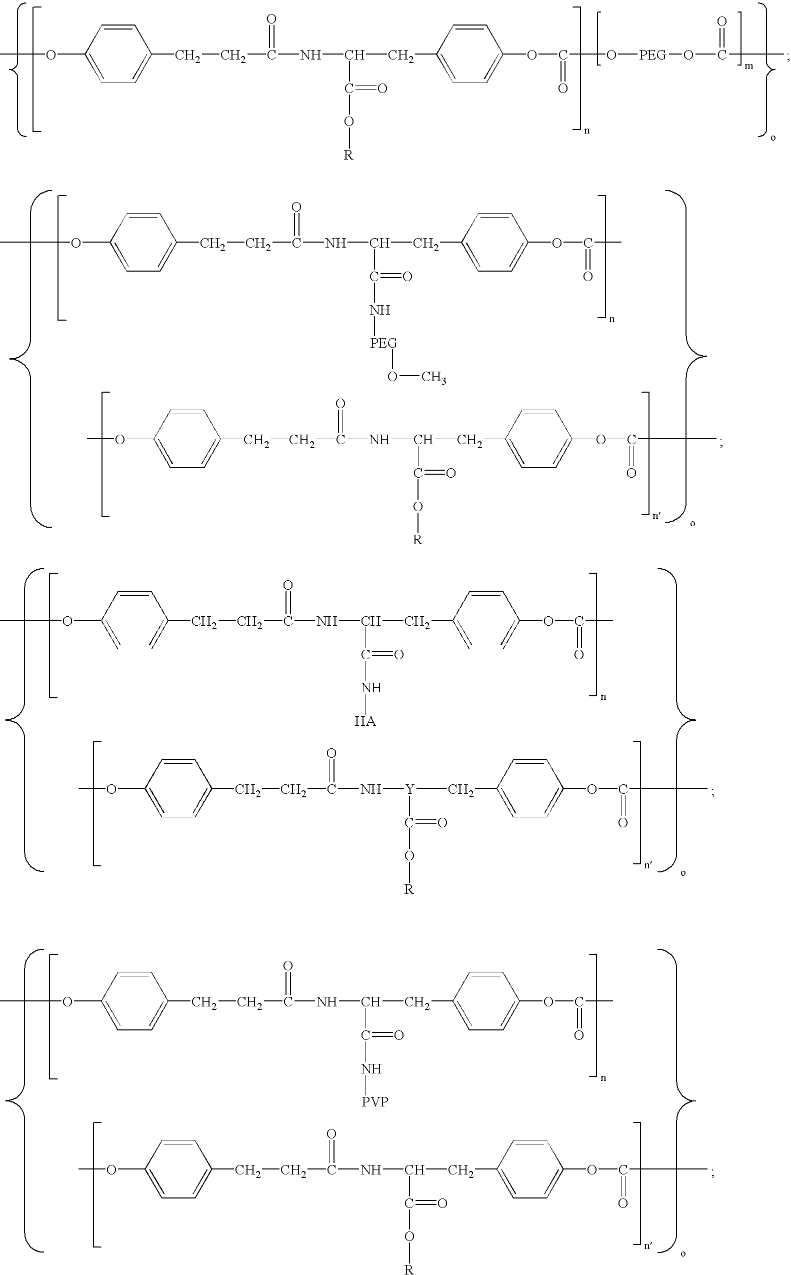 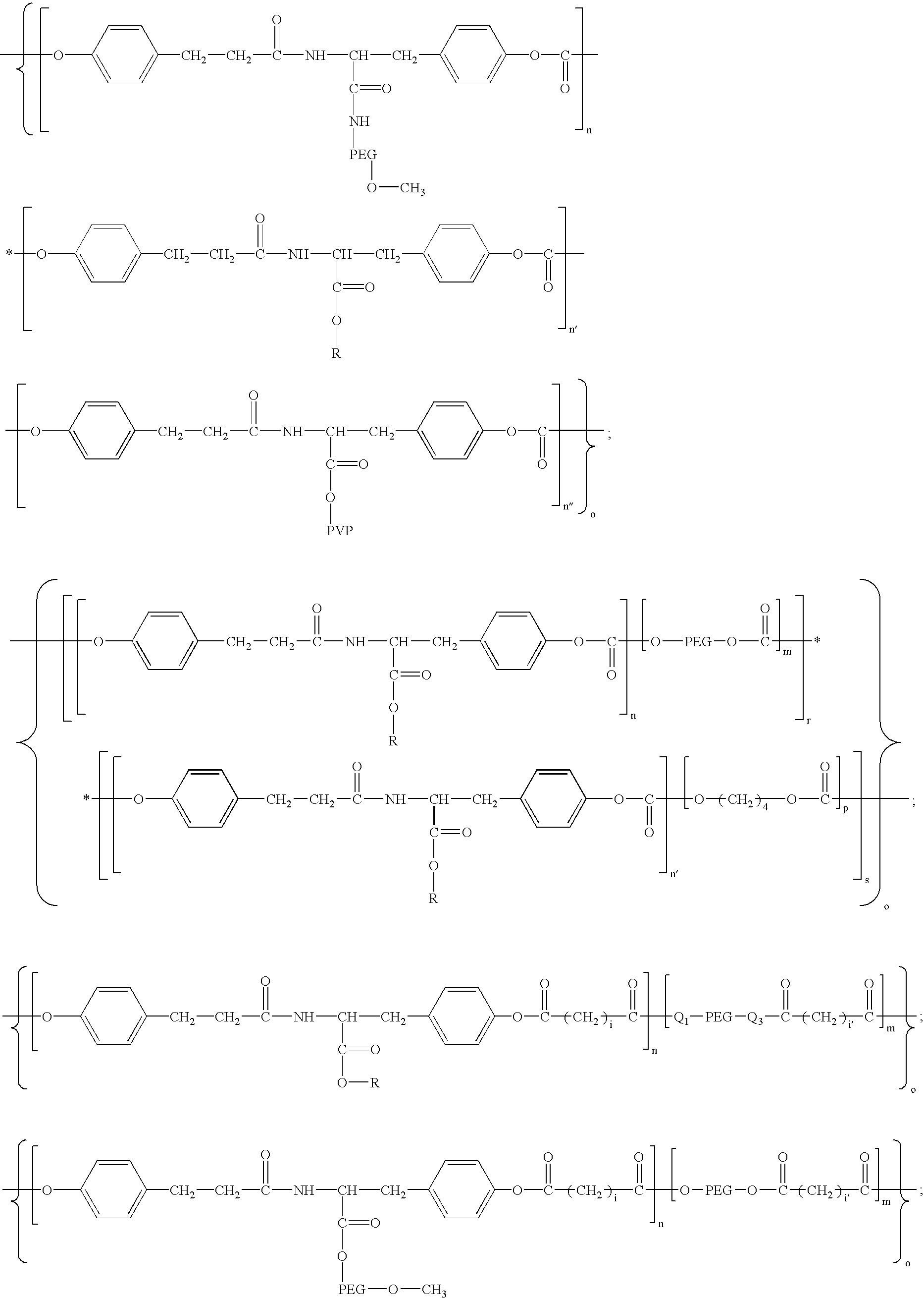 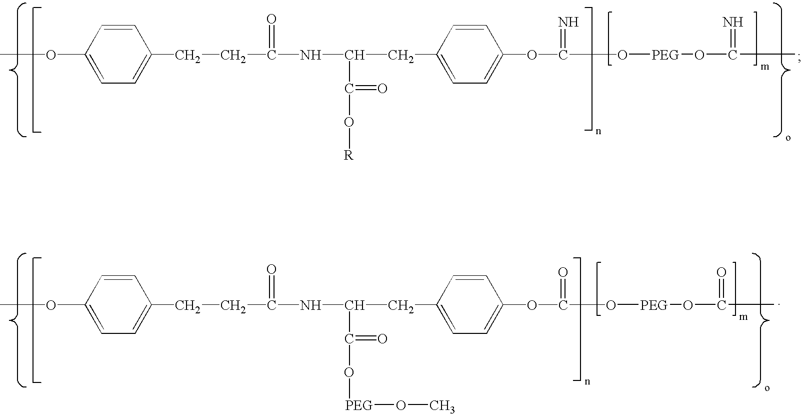
o is 4 to 3000;
m is 0.005 to 0.995;
p is 0.1 to 0.99;
i is 0 to 20;
i′ is 0 to 20;
n, n′, n″ are independently 0.005 to 0.990; and
s is 0.25 to 0.99.

15. The polymer of claim 14 with the formula:

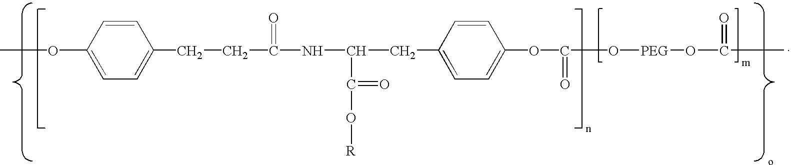

16. The polymer of claim 14 with the formula:

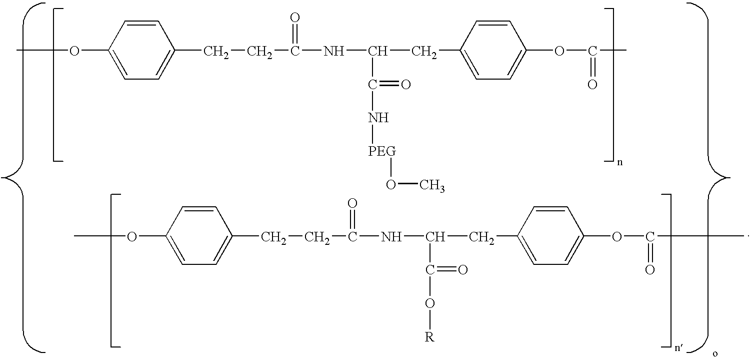

17. The polymer of claim 14 with the formula:

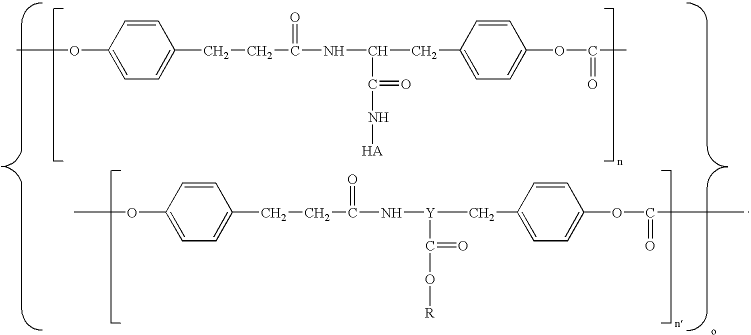

18. The polymer of claim 14 with the formula:

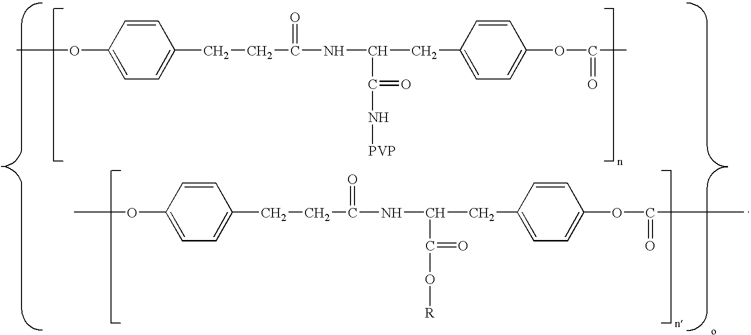

19. The polymer of claim 14 with the formula:

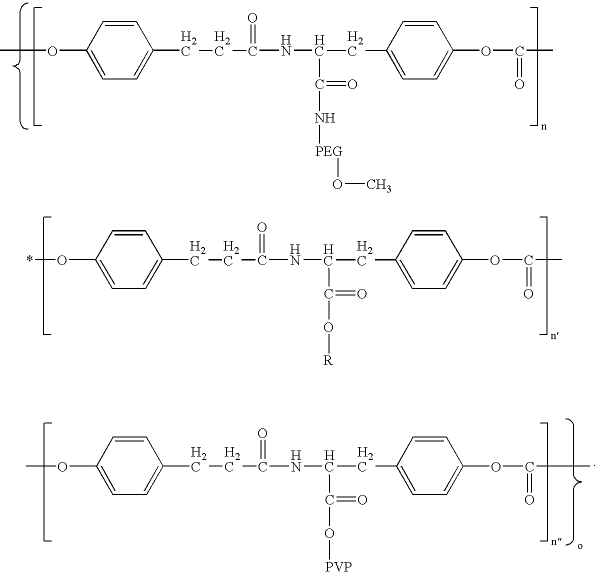

20. The polymer of claim 14 with the formula:

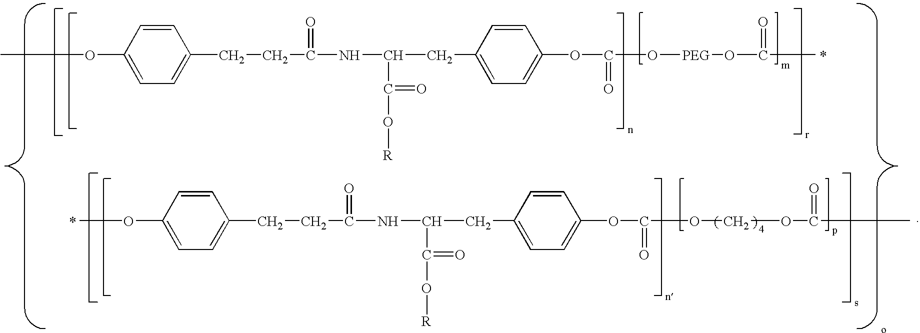

21. The polymer of claim 14 with the formula:

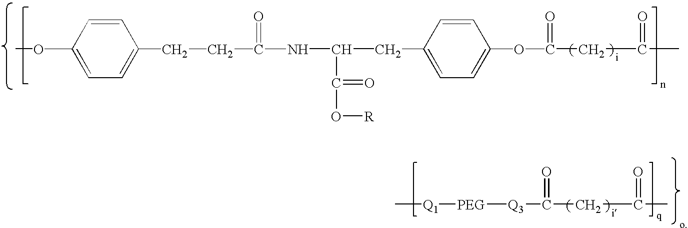

22. The polymer of claim 14 with the formula:

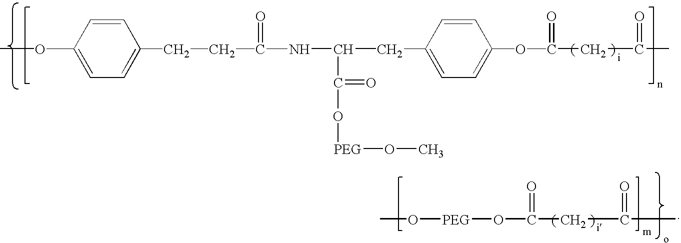

23. The polymer of claim 14 with the formula:

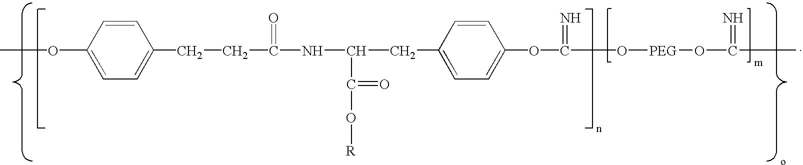

24. The polymer of claim 14 with the formula:

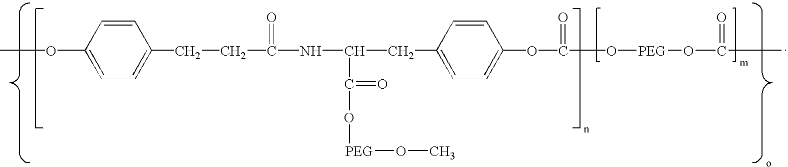

25. A medical device coating comprising a first type-one polymer, wherein a type-one polymer is a polymer as described in claim 1.

26. The medical device coating of claim 25 further comprising a second type-one polymer.

27. The medical device coating of claim 26 wherein
the second type-one polymer is disposed on the first type-one polymer; or
the first type-one polymer and the second type-one polymer are mixed together.

28. The medical device coating of claim 27 wherein the coating comprises a primer layer.

29. The medical device coating of claim 25 further comprising a type-two polymer, wherein type-two polymers are biocompatible polymers.

30. The medical device coating of claim 29 wherein type-two polymers comprise at least one of ABS resins; acrylic polymers and copolymers; acrylonitrile-styrene copolymers; alkyd resins; biomolecules; cellulose ethers; celluloses; copoly(ether-esters) (e.g. PEO/PLA); copolymers of vinyl monomers with each other and olefins; cyanoacrylates; epoxy resins; ethylene-a-olefin copolymers; ethylene-methyl methacrylate copolymers; ethylene-vinyl acetate copolymers; poly(amino acids); poly(anhydrides); poly(ester amides); poly(imino carbonates); poly(orthoesters); poly(ester amides); poly(tyrosine arylates); poly(tyrosine derive carbonates); polyalkylene oxalates; polyamides; polyanhydrides; polycarbonates; polyesters; polyethers; polyimides; polyolefins; polyorthoester; polyoxymethylenes; polyphosphazenes; polyphosphoester; polyphosphoester urethane; polyurethanes; polyvinyl aromatics; polyvinyl esters; polyvinyl ethers; polyvinyl ketones; polyvinylidene fluoride; silicones; starches; vinyl halide polymers and copolymers; other biobeneficial polymers; or their combinations.

31. The medical device coating of claims 29 wherein type-two polymers comprise at least one of poly(butyl methacrylates); poly(alkoxy acrylates); poly(alkoxy methacrylates); carboxymethyl cellulose; cellophane; cellulose; methyl cellulose; ethyl cellulose; cellulose acetate; hydroxyethyl cellulose; hydroxypropyl cellulose; cellulose acetate butyrate; cellulose butyrate; cellulose nitrate; cellulose propionate; collagen; ethylene vinyl alcohol copolymer; poly(vinyl alcohol); fibrin; fibrinogen; hyaluronic acid; Nylon 66; poly(L-lactide); poly(L-lactic acid), poly(D-lactide), poly(D-lactic acid), poly(D,L-lactic acid), poly(glycolide); poly(L-lactide-co-glycolide); poly(D,L-lactide-co-glycolide); poly(caprolactone), poly(L-lactide-co-caprolactone); poly(D,L-lactide-co-caprolactone); polydioxanone; poly(trimethylene carbonate); poly(3-hydroxy valerate); poly(3-hydroxybutyrate); poly(4-hydroxybutyrate); poly(D,L-lactic acid); poly(D,L-lactide); poly(D,L-lactide-co-glycolide); poly(D,L-lactide-co-L-lactide); poly(dioxanone); poly(glycolic acid); poly(glycolic acid-co-trimethylene carbonate); poly(glycolide); poly(hydroxybutyrate); poly(hydroxybutyrate-co-hydroxyvalerate); poly(hydroxybutyrate-co-valerate); poly(hydroxyvalerate); poly(iminocarbonate); poly(lactide-co-glycolide); poly(L-lactic acid); poly(L-lactide); poly(trimethylene carbonate); polyacrylonitrile; polycaprolactam; polycaprolactone; polydioxanone; polyisobutylene; polystyrene; styrene-ethylene/butylene-styrene triblock copolymers; styrene-isobutylene-styrene triblock copolymers; poly(vinylidene fluoride-co-chlorotrifluoroethylene); poly(vinylidene fluoride-co-hexafluoropropylene); poly(vinyl fluoride); polyvinyl acetate; PEG; POLYACTIVE; or their combinations.

32. The medical device coating of claim 29 wherein
the type-two polymer is disposed on the type-one polymer;
the type-one polymer is disposed on the type-two polymer; or
the type-one polymer and the second type-one polymer are mixed together.

33. The medical device coating of claim 32 wherein the coating comprises a primer layer.

34. The medical device coating of claim 32 further comprising a therapeutic agent.

35. The medical device coating of claim 33 further comprising a therapeutic agent.

36. The medical device of claim 34 wherein the therapeutic agent selected from proteins, peptides, antiproliferatives, antineoplastics, antiinflammatories, antiplateletes, anticoagulants, antifibrins, antithrombins, antimitotics, antibiotics, antioxidants, and their mixtures.

37. A medical device comprising the coating of claim 25.

38. A medical device comprising the coating of claim 27.

39. A medical device comprising the coating of claim 28.

40. A medical device comprising the coating of claim 29.

41. The medical device of claims 40 wherein type-two polymers comprise at least one of ABS resins; acrylic polymers and copolymers; acrylonitrile-styrene copolymers; alkyd resins; biomolecules; cellulose ethers; celluloses; copoly(ether-esters) (e.g. PEO/PLA); copolymers of vinyl monomers with each other and olefins; cyanoacrylates; epoxy resins; ethylene-a-olefin copolymers; ethylene-methyl methacrylate copolymers; ethylene-vinyl acetate copolymers; poly(amino acids); poly(anhydrides); poly(ester amides); poly(imino carbonates); poly(orthoesters); poly(ester amides); poly(tyrosine arylates); poly(tyrosine derive carbonates); polyalkylene oxalates; polyamides; polyanhydrides; polycarbonates; polyesters; polyethers; polyimides; polyolefins; polyorthoester; polyoxymethylenes; polyphosphazenes; polyphosphoester; polyphosphoester urethane; polyurethanes; polyvinyl aromatics; polyvinyl esters; polyvinyl ethers; polyvinyl ketones; polyvinylidene fluoride; silicones; starches; vinyl halide polymers and copolymers; other biobeneficial polymers; or their combinations.

42. The medical device of claim 37 further comprising a therapeutic agent.

43. The medical device of claim 40 further comprising a therapeutic agent.

44. The medical device of claim 42 wherein the therapeutic agent selected from proteins, peptides, aantiproliferatives, antineoplastics, antiinflammatories, antiplateletes, anticoagulants, antifibrins, antithrombins, antimitotics, antibiotics, antioxidants, and their mixtures.

45. The medical device of claim 44 wherein the device is selected from self-expandable stents, balloon-expandable stents, stent-grafts, venous, arterial, or aortic grafts, vascular grafts, artificial heart valves, closure devices for patent foramen ovale, cerebrospinal fluid shunts, pacemaker electrodes, guidewires, ventricular assist devices, artificial hearts, cardiopulmonary by-pass circuits, blood oxygenators, and endocardial leads.

46. A method of making the polymer of claim 25 comprising providing
an appropriate amount of A-moiety with the formula 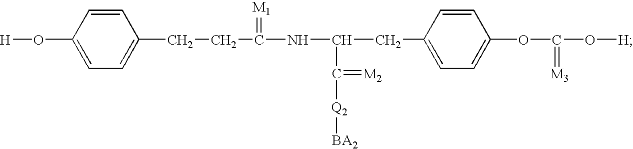
a B-moiety with the formula 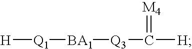
a C-type moiety, diol or diamine diradical, with the formula
H—O—R₃—O—H; or
a D-type moiety, diacid radical, with the formula 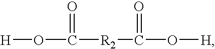
wherein
M₁-M₄ are independently selected from O, NH, CH₂, or S;
Q₁-Q₃ are independently selected from Group-15- or Group-16-containing moieties;
BA₁ and BA₂ are independently selected from an R-group or a bioactive moiety, wherein the R-group is a C₁-C₂₀, linear or branched, (un)substituted alkyl or aryl and provided that at least 0.001 mole percent of the total of BA₁ and BA₂ are selected from one or more bioactive moieties.

47. The polymer of claim 1 wherein diamine diradical comprises NH₂—(CH₂)₂₋₅—NH₂.
